(19) United States
(12) Patent Application Publication
(10) Pub. No.: US 2025/0197962 A1
(43) Pub. Date: Jun. 19, 2025

(54) MATERIALS AND METHODS FOR RECOVERING METALS FROM ORE

(71) Applicant: Brokkr Mineral Resources Corporation, Burnaby (CA)

(72) Inventors: Sean CROWE, Burnaby (CA); Robert Crandall GREENE, Burnaby (CA)

(21) Appl. No.: 18/982,877

(22) Filed: Dec. 16, 2024

Related U.S. Application Data
(63) Continuation of application No. PCT/US2023/068612, filed on Jun. 16, 2023.
(60) Provisional application No. 63/352,672, filed on Jun. 16, 2022.

(51) Int. Cl. C22B 3/18; C22B 3/00; C22B 47/00

(52) U.S. Cl. CPC C22B 3/18; C22B 23/0407; C22B 47/00

(57) ABSTRACT

The present disclosure relates to methods and materials for leaching and recovering metals from oxide ores.

### CROSS-REFERENCE TO RELATED APPLICATIONS

This application claims the benefit of priority to U.S. Provisional Application No. 63/352,672, filed Jun. 16, 2022, which is incorporated herein by reference in its entirety.

### REFERENCE TO AN ELECTRONIC SEQUENCE LISTING

The contents of the electronic sequence listing (3RCS_002_02WO_SeqList_ST26.xml; Size: 7,030 bytes; and Date of Creation: Jun. 16, 2023) are herein incorporated by reference in its entirety.

### BACKGROUND OF THE INVENTION

Ore obtained from a mine site typically comprises one or more metals of commercial value. For example, laterite ore contains nickel and cobalt, which are metals that can attract a high price on the commodities market due to their industrial applications. The primary uses of nickel include the production of stainless steel, rechargeable, nickel cobalt aluminum (NCA), nickel cobalt manganese (NCM), and NiCad batteries, and the production of other electronic and computer equipment.

In some ores, however, the metal is present in a stable form or in very small amounts, which can make the removal process difficult and expensive. This is the case with nickel laterites, which are highly stable and contain relatively low levels of nickel and cobalt. In addition, most reserves cannot be concentrated, thus requiring all of the ore to be processed to extract the nickel and/or cobalt. As a result of these and other challenges, current processes to remove nickel and cobalt from such ores, particularly on industrial scale, rely on aggressive processing treatments that amplify the costs, environmental footprints, and increase the technical difficulties.

Accordingly, in the absence of improved technologies, there is widespread concern in the industry about the technical and economic viability of nickel laterite and other oxide ore processing.

The present invention addresses these and other unmet needs.

### SUMMARY

In general, the present disclosure relates to a new method for recovering nickel and/or cobalt from an oxide ore by a process that comprises the reductive dissolution of ferric oxide minerals. The present disclosure also relates to a new method for extracting and recovering nickel and/or cobalt from an oxide ore by a process that comprises the reductive dissolution of ferric oxide minerals.

In some embodiments, the present disclosure provides a process for leaching nickel and/or cobalt from an oxide ore comprising iron, the process comprising: (a) combining (i) one or more microorganisms, (ii) a substrate mixture, (iii) the oxide ore, (iv) an optional ligand, and (v) an aqueous phase to form a mixture; (b) maintaining the mixture of step a) for a pre-determined period of time under conditions sufficient to at least partially reduce an amount of ferric iron in the ore to ferrous iron, wherein the ferrous iron and one or more metals are solubilized in the aqueous phase; and (c) isolating the aqueous phase as a leach solution comprising the ferrous iron and an amount of dissolved nickel and/or cobalt, from the mixture.

In some embodiments, the one or more microorganisms are selected from the group consisting of Thermincola potens JR (NC_014152.1), Listeria monocytogenes (NC_003210.1), Clostridium celerecrescens (NZ_PGET01000001.1), Bacillus infernus, Bacillus subterraneus, Bacillus pseudormus MC02, Bacillus subterraneus, Thermoanaerobacter siderophilus (NZ_CM001486.1), Carboxydothermus ferrireducens (NZ_ATYG01000001.1), Carboxydothermus siderophilus, Carboxydothermus pertinax (NZ_BDJK01000055.1), Carboxydocella thermautotrophica (NZ_CP028491.1), Moorella humiferrea (NZ_PVXM01000006.1), Geosporobacter ferrireducens IRF9, Thermotalea metallivorans B2-1(T), Pelosinus fermentans, Thermotoga maritima (NC_023151.1), Sinirhodobacter ferrireducens (NZ_SAVB01000001.1), Acidiphilium SJH, Acidiphilium PK40, Acidiphilium PK46, Acidiphilium KPWI4, Acidiphilium cryptum JF-5, Acidocella facilis, Acidocella M21, Acidocella PFBC, Acidocella PWB4, Shewanella putrefaciens (NZ_CP066370.1), Shewanella oneidensis MR-1 (NC_004347.2), Shewanella alga strain BrY (NZ_CP046378.1), Shewanella amazonensis sp. nov., Shewanella putrefaciens IR-1, Shewanella sp. HN-41, Shewanella putrefaciens CN-32, Shewanella putrefaciens 200R, Shewanella oneidensis MR-1, Shewanella baltica W3-6-1, Shewanella sp.PV-4, Shewanella peizotolerans WP3, Shewanella decolorationis, Shewanella frigidimarina, Shewanella gelidimarina, Shewanella loihica, Shewanella pealeana, Serratia plymuthica (NC_015567.1), Serratia fonticola (NZ_CP011254.1), Aeromonas hydrophila (NZ_CP050851.1), Klebsiella oxytoca (NZ_CP033844.1), Ferrimonas balearica (NC_014541.1), Frateuria-like isolate WJ2, Desulfovibrio ferrophilus (NZ_AP017378.1), Desulfuromusa ferrireducens, Desulfuromonas svalbardensis, Desulfuromonas acetoxidans (JABWTG01), Geobacter sulfurreducens (NC_002939.5), Geobacter bemidjiensis (NC_011146.1), Geobacter metallireducens (NC_007517.1), Geobacter pelophilus strain Dfr2 (NZ_JAHCVJ010000001.1), Geobacter daltonii (NC_011979.1), Geobacter chapellei (NZ_JAHDYS010000001.1), Geobacter psychrophilus sp. nov, Geobacter bremensis sp. nov; Geobacter lovleyi sp. nov. strain SZ, Geobacter luticola, Geobacter pickeringii, Geobacter argillaceus, Geobacter uraniireducens, Desulfosediminicola ganghwensis (NZ_CP050699.1), Desulfosediminicola flagellatus (NZ_CP050698.1), Anaeromyxobacter dehalogenans (NC_011891.1), Anaeromyxobacter Strain FAc12, Geoalkalibacter subterraneus (NZ_CP010311.1), Geoalkalibacter ferrihydriticus (NZ_FNGU01000001.1), Rhodoferax ferrireducens (NC_007908.1), Ferribacterium limneticum (NZ_CP075189.1), Geothrix fermentans (NZ_KE386810.1), Geovibrio ferrireducens, Deferribacter thermophilus, Pyrobaculum islandicum (NC_008701.1), Pyrodictium abyssi, Methanopyrus kandleri (NC_003551.1), Archaeoglobus fulgidus (NZ_CP006577.1), Pyrococcus furiosus (NZ_CP023154.1), Methanococcus thermolithotrophicus (NZ_AQXV01000055.1), Ferroglobus placidus (NC_013849.1), Geoglobus acetivorans sp. nov., Acidobacterium capsulatum, Acidobacterium PK35, Acidobacterium RIT23, Acidobacterium WJ7, Telmatospirillum (Alphaproteobacterial Genus) (PRJNA561022), Sideroxydans lithotrophicus ES-1, Magnetospirillum magneticum AMB-1, Candidatus Tectomicrobia, Candidatus Zixibacteria, Candidatus Tectomicrobia, Candidatus Dadabacteria, Candidatus Handelsmanbacteria, Carboxydothermus hydrogenoformans, Carboxydothermus islandicus, Telmatospirillum siberiense, Nitrosococcus halophilus, Skermanella stibiresistens, Insolitispirillum peregrinum, Magnetospirillum magneticum, and mixtures thereof. In some embodiments, the one or more microorganisms are genetic mutants thereof. In some embodiments, the one of more microorganisms is a Shewanella spp. In some embodiments, the one or more microorganisms is Shewanella putrefaciens IR-1, Shewanella putrefaciens CN-32, or Shewanella putrefaciens 200R In some embodiments, the one or more microorganisms is Shewanella putrefaciens 200R In some embodiments, the one or more microorganisms is Shewanella oneidensis MR-1.

In some embodiments, the one or more microorganisms is transgenic. In some embodiments the one or more microorganisms is transgenic and modified to express foreign genes that code for outer-membrane cytochromes. In some embodiments the one or more microorganisms is transgenic and modified to express foreign genes that code for OmcS. In some embodiments the one or more microorganisms is transgenic and modified to express foreign genes that code for OmcS from Geobacter sulfurrreducens. In some embodiments, the transgenic and modified organism is a Shewanella spp. In some embodiments, the one or more microorganisms is Shewanella oneidensis MR-1 modified to express OmcS. In some embodiments, the one or more microorganisms is Shewanella oneidensis MR-1 modified to heterologously express OmcS and one or more genes that enhance flavin production. In some embodiments, the one or more microorganisms is Shewanella oneidensis strain C5, which is modified to heterologously express the Bacillus subtilis genes ribAEDHC. In some embodiments, the one or more microorganisms is Shewanella oneidensis strain C5 modified to express OmcS. In some embodiments, the C5 modified strain is the C5-OS₁, C5-OS₂, or C5-OS₃ strain disclosed in Lin, T., et al. “Simultaneous heterologous expression of cytochrome OmcS and enhanced flavin biosynthesis to enhance extracellular electron exchange in Shewanella oneidensis”, SSRN electronic journal, posted Apr. 13, 2022, which is incorporated herein by reference in its entirety.

In some aspects, the process further comprises: (d) contacting the leach solution of step (c) with a conjugate comprising a peptide and a polystyrene (PS) bead for a period of time under conditions that form a conjugate-metal ion complex, wherein the peptide is configured to selectively bind nickel and/or cobalt; (e) isolating the conjugate-metal ion complex of step (d); (f) eluting the metal ion(s) from the conjugate-metal ion complex by washing the complex with acid, thereby forming a solution; (g) transferring the solution of step (f) comprising the metal ions to an electrowinning circuit; (h) applying an electric current through the solution of step (f) comprising the metal ions; (i) collecting the resulting metal(s) on a surface of a cathode; and (j) recovering the metal(s) from the cathode.

In some embodiments, the substrate mixture comprises a reducing agent. In some embodiments, the substrate mixture comprises carbonaceous waste products, elemental sulfur, substances containing partially reduced sulfur forms, or mixtures thereof. In some embodiments, the substrate mixture comprises molasses, sugar, beet sugar, cane sugar, dextrose hydrolyzed from corn, dextrose hydrolyzed from cornstarch, sucrose containing materials, fermentation products thereof, or mixtures thereof.

In some embodiments, the oxide ore is a laterite ore. In some embodiments, the oxide ore is a nickel laterite ore. In some embodiments, the oxide ore comprises about 0.1% to about 15% by weight nickel. In some embodiments, the oxide ore comprises about 0.1% to about 2% by weight cobalt. In some embodiments, the oxide ore further comprises chromium and/or manganese. In some embodiments, the oxide ore further comprises rare earth elements, and/or scandium, and/or platinum group elements. In some embodiments, the oxide ore further comprises rare earth elements and/or platinum group elements.

In some embodiments, the mixture of step (a) further comprises a ligand. In some embodiments, the ligand is an organic acid or combination of organic acids. In some embodiments, the ligand is an organic acid selected from the group consisting of malic acid, lactic acid, gluconic acid, pyruvic acid, succinic acid, ketoglutaric acid, oxalic acid, fumaric acid, citric acid, acetic acid, malonic acid, salicylic acid, acetohydroxamic acid, mugineic acid, benzoic acid, hydroxamic acid, and a combination thereof. In some embodiments, the ligand is an organic acid selected from the group consisting of malic acid, lactic acid, gluconic acid, pyruvic acid, succinic acid, ketoglutaric acid, oxalic acid, fumaric acid, citric acid, and a combination thereof.

In some embodiments, the mixture of step (a) further comprises an electron shuttle. In some embodiments, the electron shuttle is an organic compound or combination of organic compounds. In some embodiments, the electron shuttle is an organic compound selected from the group consisting of flavins (including compounds that contain tricyclic heterocycle isoalloxazine or its isomer alloxazine, and derivatives thereof), quinones, humic acids, fulvic acids, and biochar, or a combination thereof.

In some embodiments, the aqueous phase of step (a) comprises a buffer to maintain an appropriate pH level. In some embodiments, the buffer maintains a pH of about 5 to 9. In some embodiments, the buffer maintains a pH of about 5 to 8.

The present invention also provides conjugates comprising a peptide and a polystyrene (PS) bead. In some embodiments, the peptide comprises the structure: —NH-X_{AA1}-X_{AA2}-His-, wherein X_{AA1} and X_{AA2} are each independently an amino acid residue other than histidine. In some embodiments, the peptide comprises the structure: —NH-X_{AA1}-X_{AA2}-His-X_{AA3}, wherein X_{AA1}, X_{AA2}, and X_{AA3} are each independently an amino acid residue other than histidine. In some embodiments, X_{AA1} and X_{AA2} are each independently a residue of a natural or unnatural amino acid. In some embodiments, X_{AA1} and X_{AA2} are each independently a residue of a natural amino acid. In some embodiments, X_{AA1} and X_{AA2} are each independently a residue of glycine, alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or a-hydroxymethylserine. In some embodiments, —NH-X_{AA1}-X_{AA2}-His- is selected from the group consisting of: -HN-DAH, -HN-DTH, -HN-VIH, -HN-MDH, -HN-RFH, -HN-RTH, -HN-HSH, -HN-GGH, -HN-GKH, -HN-KGH, -HN-KKH, -HN-YYH, -HN-MNH, -HN-kGH, -HN-kGH, -HN-GkH, and -HN-kkH. In some embodiments, X_{AA3} is a lysine residue

In some embodiments, the conjugate of the present disclosure has the structure:

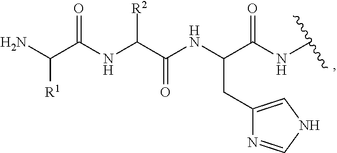

wherein R¹ and R² are each independently H or a side chain of an amino acid. In some embodiments, the side chain is positively charged. In some embodiments, the side chain comprises an amine or guanidine moiety. In some embodiments, R¹ and R² are each independently the side chain of a lysine, ornithine, arginine, or homoarginine residue.

In some embodiments, the peptides of the present disclosure are configured to selectively bind nickel. In some embodiments, the peptide has a selectivity for nickel over iron that is greater than 3:1, greater that 5:1, greater that 7:1, or greater than 10:1. In some embodiments, the peptide has a binding affinity for Ni^{II} of from about 1×10⁻¹⁶ to about 1×10⁻⁸ M⁻¹.

In some embodiments, the peptides of the present disclosure are configured to selectively bind cobalt. In some embodiments, the peptide has a selectivity for cobalt over iron that is greater than 3:1, greater that 5:1, greater that 7:1, or greater than 10:1. In some embodiments, the peptide has a binding affinity for Co^{II} of from about 1×10⁻¹⁶ to about 1×10⁻⁸ M⁻¹.

In some embodiments, the process further comprises the removal of iron from the leach solution through a combination of aeration, pH adjustments, and/or selective precipitation.

### BRIEF DESCRIPTION OF THE DRAWINGS

FIG. 1A-1D shows the short-term reduction of laterite-derived materials (FIG. 1A, Fe;

FIG. 1B, Ni; FIG. 1C, Mn; FIG. 1D, Co) by Shewanella putrefaciens 200R, in batch reactors (with and without citrate amendment) according to a leaching process of the present disclosure.

FIG. 2 shows the long-term reduction of laterite derived materials by Shewanella putrefaciens 200R, in flow-through reactors amended with citrate according to a leaching process of the present disclosure.

FIG. 3 is a schematic showing non-limiting leaching circuit options of the present disclosure.

FIG. 4A is a schematic showing a synthesis of disclosed conjugates (“IX beads”) comprising a peptide and PEG-coating polystyrene (PS bead). FIG. 4B shows the complexation of Ni²⁺ ions with a conjugate of the present disclosure obtained by contacting the conjugate with the metal ions dissolved in a pregnant leach solution.

FIGS. 5A-5G provide flowcharts of non-limiting processes encompassed by the present disclosure for recovering one or more metals from an oxide ore.

### DETAILED DESCRIPTION

### Definitions

The term “about” when immediately preceding a numerical value means± up to 20% of the numerical value. For example, “about” a numerical value means± up to 20% of the numerical value, in some embodiments, ± up to 19%, ± up to 18%, ± up to 17%, ± up to 16%, ± up to 15%, ± up to 14%, ± up to 13%, ± up to 12%, ± up to 11%, ± up to 10%, ± up to 9%, ± up to 8%, ± up to 7%, ± up to 6%, ± up to 5%, ± up to 4%, ± up to 3%, ± up to 2%, ± up to 1%, ± up to less than 1%, or any other value or range of values therein.

Throughout the present specification, numerical ranges are provided for certain quantities. These ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “heap” as used herein, includes a heap, a dump, a pit, a vat, or a column which contains an ore that is to be processed.

The term “pregnant aqueous solution” or “pregnant leach solution” as used herein refers to an aqueous solution containing one or more dissolved target metals.

### Leaching Processes

In some embodiments, the present disclosure provides a bioleaching process for recovering one or more metals (e.g., nickel and/or cobalt) from an oxide ore comprising iron, wherein the process comprises: (a) combining (i) one or more microorganisms, (ii) a substrate mixture, (iii) the oxide ore, (iv) an optional ligand, and (v) an aqueous phase to form a mixture; (b) maintaining the mixture of step (a) for a pre-determined period of time under conditions sufficient to at least partially reduce an amount of ferric iron in the ore to ferrous iron, wherein the ferrous iron and nickel are solubilized in the aqueous phase; and (c) isolating the aqueous phase as a pregnant leach solution comprising the ferrous iron and an amount of one or more dissolved metals, from the mixture.

In some embodiments, the present disclosure provides a bioleaching process for recovering one or more metals (e.g., nickel and/or cobalt) from an oxide ore comprising iron, wherein the process comprises: (a) combining (i) one or more microorganisms, (ii) a substrate mixture, (iii) the oxide ore, (iv) an optional ligand, (v) optional electron shuttles, and (vi) an aqueous phase to form a mixture; (b) maintaining the mixture of step (a) for a pre-determined period of time under conditions sufficient to at least partially reduce an amount of ferric iron in the ore to ferrous iron, wherein the ferrous iron and nickel are solubilized and/or stabilized in the aqueous phase; and (c) isolating the aqueous phase as a pregnant leach solution comprising the ferrous iron and an amount of one or more dissolved metals, from the mixture.

In some embodiments, the present disclosure provides a process for leaching one or more metals (e.g., nickel and/or cobalt) from an oxide ore comprising iron, wherein the process comprises: (a) combining (i) one or more microorganisms, (ii) a substrate mixture, (iii) the oxide ore, (iv) an optional ligand, and (v) an aqueous phase to form a mixture; (b) maintaining the mixture of step (a) for a pre-determined period of time under conditions sufficient to at least partially reduce an amount of ferric iron in the ore to ferrous iron, wherein the ferrous iron and nickel are solubilized in the aqueous phase; and (c) isolating the aqueous phase as a pregnant leach solution comprising the ferrous iron and an amount of one or more dissolved metals, from the mixture.

In some embodiments, the present disclosure provides a process for leaching one or more metals (e.g., nickel and/or cobalt) from an oxide ore comprising iron, wherein the process comprises: (a) combining (i) one or more microorganisms, (ii) a substrate mixture, (iii) the oxide ore, (iv) an optional ligand, (v) an optional electron shuttle, and (vi) an aqueous phase to form a mixture; (b) maintaining the mixture of step (a) for a pre-determined period of time under conditions sufficient to at least partially reduce an amount of ferric iron in the ore to ferrous iron, wherein the ferrous iron and nickel are solubilized and/or stabilized in the aqueous phase; and (c) isolating the aqueous phase as a pregnant leach solution comprising the ferrous iron and an amount of one or more dissolved metals, from the mixture.

In some embodiments, the present disclosure provides a process for leaching one or more metals from an oxide ore comprising iron, wherein the process comprises: (a) contacting the oxide ore with a leaching solution comprising (i) one or more microorganisms, (ii) a substrate mixture, (iii) an optional ligand, and (iv) an aqueous phase to form a mixture; (b) maintaining the mixture of step (a) for a pre-determined period of time under conditions sufficient to at least partially reduce an amount of ferric iron in the ore to ferrous iron, wherein the ferrous iron and nickel are solubilized in the aqueous phase; and (c) isolating the aqueous phase as a leach solution comprising the ferrous iron and an amount of one or more dissolved metals, from the mixture.

In some embodiments, the present disclosure provides a process for leaching one or more metals from an oxide ore comprising iron, wherein the process comprising: (a) contacting the oxide ore with a leaching solution comprising (i) one or more microorganisms, (ii) a substrate mixture, (iii) an optional ligand, (iv) an optional electron shuttle, and (v) an aqueous phase to form a mixture; (b) maintaining the mixture of step (a) for a pre-determined period of time under conditions sufficient to at least partially reduce an amount of ferric iron in the ore to ferrous iron, wherein the ferrous iron and nickel are solubilized in the aqueous phase; and (c) isolating the aqueous phase as a leach solution comprising the ferrous iron and an amount of one or more dissolved metals, from the mixture.

In some embodiments, the one or more metals in the ore are nickel and/or cobalt. In some embodiments, the one or more metals in the ore is nickel. In some embodiments, the one or more metals in the ore is cobalt. In some embodiments, the oxide ore further comprises copper. In some embodiments, the oxide ore further comprises chromium and/or manganese.

### Microorganisms

The microorganisms for use in the processes disclosed herein can be any microorganism or combinations of microorganisms that promotes or accelerates the reductive dissolution of ferric iron minerals in oxide ores, which results in the associated metals (e.g., nickel and/or cobalt) being released from the mineral and into solution. The microorganisms can be found in the ore (sometimes referred to as biostimulation) and/or may be added to the ore (sometimes referred to as bioaugmentation) to further promote or accelerate reductive dissolution, as desired.

In some embodiments, the one or more microorganisms produce an enzyme or comprise an enzyme system selected from the group consisting of CymA, DFE, DmkA/DmkB, EetA/EetB, FmnA/FmnB, GACE, MtrA/MtrA/MtrC, Ndh2, OmcF/OmcS/OmcZ, PplA, and T4ap, or combinations thereof. In a specific embodiment, the microorganisms may comprise a mutant form of CymA, DFE, DmkA/DmkB, EetA/EetB, FmnA/FmnB, GACE, MtrA/MtrA/MtrC, Ndh2, OmcF/OmcS/OmcZ, PplA, and T4ap, or combinations thereof.

In some embodiments, the one or more microorganisms is a bacterial and/or fungal species that is capable of reducing iron. In some embodiments, the one or more microorganisms is a bacterial species that is capable of reducing iron. In some embodiments, the one or more microorganisms is a bacterial species that catalyzes the reduction of ferric iron to ferrous iron.

In some embodiments, the one or more microorganisms are selected from the group consisting of Thermincola potens JR (NC_014152.1), Listeria monocytogenes (NC_003210.1), Clostridium celerecrescens (NZ_PGET01000001.1), Bacillus infernus, Bacillus subterraneus, Bacillus pseudormus MC02, Bacillus subterraneus, Thermoanaerobacter siderophilus (NZ_CM001486.1), Carboxydothermus ferrireducens (NZ_ATYG01000001.1), Carboxydothermus siderophilus, Carboxydothermus pertinax (NZ_BDJK01000055.1), Carboxydocella thermautotrophica (NZ_CP028491.1), Moorella humiferrea (NZ_PVXM01000006.1), Geosporobacter ferrireducens IRF9, Thermotalea metallivorans B2-1(T), Pelosinus fermentans, Thermotoga maritima (NC_023151.1), Sinirhodobacter ferrireducens (NZ_SAVB01000001.1), Acidiphilium SJH, Acidiphilium PK40, Acidiphilium PK46, Acidiphilium KPWI4, Acidiphilium cryptum JF-5, Acidocella facilis, Acidocella M21, Acidocella PFBC, Acidocella PWB4, Shewanella putrefaciens (NZ_CP066370.1), Shewanella oneidensis MR-1 (NC_004347.2), Shewanella alga strain BrY (NZ_CP046378.1), Shewanella amazonensis sp. nov., Shewanella putrefaciens IR-1, Shewanella sp. HN-41, Shewanella putrefaciens CN-32, Shewanella putrefaciens 200R, Shewanella oneidensis MR-1, Shewanella baltica W3-6-1, Shewanella sp.PV-4, Shewanella peizotolerans WP3, Shewanella decolorationis, Shewanella frigidimarina, Shewanella gelidimarina, Shewanella loihica, Shewanella pealeana, Serratia plymuthica (NC_015567.1), Serratia fonticola (NZ_CP011254.1), Aeromonas hydrophila (NZ_CP050851.1), Klebsiella oxytoca (NZ_CP033844.1), Ferrimonas balearica (NC_014541.1), Frateuria-like isolate WJ2, Desulfovibrio ferrophilus (NZ_AP017378.1), Desulfuromusa ferrireducens, Desulfuromonas svalbardensis, Desulfuromonas acetoxidans (JABWTG01), Geobacter sulfurreducens (NC_002939.5), Geobacter bemidjiensis(NC_011146.1), Geobacter metallireducens (NC_007517.1), Geobacter pelophilus strain Dfr2 (NZ_JAHCVJ010000001.1), Geobacter daltonii (NC_011979.1), Geobacter chapellei (NZ_JAHDYS01000000.1), Geobacter psychrophilus sp. nov, Geobacter bremensis sp. nov; Geobacter lovleyi sp. nov. strain SZ, Geobacter luticola, Geobacter pickeringii, Geobacter argillaceus, Geobacter uraniireducens, Desulfosediminicola ganghwensis (NZ_CP050699.1), Desulfosediminicola flagellatus (NZ_CP050698.1), Anaeromyxobacter dehalogenans (NC_011891.1), Anaeromyxobacter Strain FAc12, Geoalkalibacter subterraneus (NZ_CP010311.1), Geoalkalibacter ferrihydriticus (NZ_FNGU01000001.1), Rhodoferax ferrireducens (NC_007908.1), Ferribacterium limneticum (NZ_CP075189.1), Geothrix fermentans (NZ_KE386810.1), Geovibrio ferrireducens, Deferribacter thermophilus, Pyrobaculum islandicum (NC_008701.1), Pyrodictium abyssi, Methanopyrus kandleri (NC_003551.1), Archaeoglobus fulgidus (NZ_CP006577.1), Pyrococcus furiosus (NZ_CP023154.1), Methanococcus thermolithotrophicus (NZ_AQXV01000055.1), Ferroglobus placidus (NC_013849.1), Geoglobus acetivorans sp. nov., Acidobacterium capsulatum, Acidobacterium PK35, Acidobacterium RIT23, Acidobacterium WJ7, Telmatospirillum (Alphaproteobacterial Genus) (PRJNA561022), Sideroxydans lithotrophicus ES-1, Magnetospirillum magneticum AMB-1, Candidatus Tectomicrobia, Candidatus Zixibacteria, Candidatus Tectomicrobia, Candidatus Dadabacteria, Candidatus Handelsmanbacteria, Carboxydothermus hydrogenoformans, Carboxydothermus islandicus, Telmatospirillum siberiense, Nitrosococcus halophilus, Skermanella stibiresistens, Insolitispirillum peregrinum, Magnetospirillum magneticum, and mixtures and/or genetic mutants, thereof.

In some embodiments, the one of more microorganisms is a Shewanella spp. In some embodiments, the one or more microorganisms is Shewanella putrefaciens IR-1, Shewanella putrefaciens CN-32, Shewanella putrefaciens 200R, or Shewanella oneidensis MR-1. In some embodiments, the one of more microorganisms is a Shewanella spp. In some embodiments, the one or more microorganisms is Shewanella putrefaciens IR-1, Shewanella putrefaciens CN-32, or Shewanella putrefaciens 200R In some embodiments, the one or more microorganisms is Shewanella putrefaciens IR-1. In some embodiments, the one or more microorganisms is Shewanella putrefaciens CN-32. In some embodiments, the one or more microorganisms is Shewanella putrefaciens 200R In some embodiments, the one or more microorganisms is Shewanella oneidensis MR-1.

In some embodiments, the one or more microorganisms is transgenic. In some embodiments the one or more microorganisms is transgenic and modified to express foreign genes that code for outer-membrane cytochromes. In some embodiments the one or more microorganisms is transgenic and modified to express foreign genes that code for OmcS. In some embodiments the one or more microorganisms is transgenic and modified to express foreign genes that code for OmcS from Geobacter sulfurrreducens. In some embodiments, the transgenic and modified organism is a Shewanella spp. In some embodiments, the one or more microorganisms is Shewanella oneidensis MR-1 modified to express OmcS. In some embodiments, the one or more microorganisms is Shewanella oneidensis MR-1 modified to heterologously express OmcS and one or more genes that enhance flavin production. In some embodiments, the one or more microorganisms is Shewanella oneidensis strain C5, which is modified to heterologously express the Bacillus subtilis genes ribAEDHC. In some embodiments, the one or more microorganisms is Shewanella oneidensis strain C5 modified to express OmcS. In some embodiments, the C5 modified strain is C5-OS₁, C5-OS₂, or C5-OS₃, which have different ribosome binding sites to control OmcS expression. These modified strains and others useful in the present invention are disclosed in Lin, T., et al. “Simultaneous heterologous expression of cytochrome OmcS and enhanced flavin biosynthesis to enhance extracellular electron exchange in Shewanella oneidensis”, SSRN electronic journal, posted Apr. 13, 2022, which is incorporated herein by reference in its entirety.

In some embodiments, the microorganism possesses one or more mutations. In some embodiments, the microorganism is mutated to enhance its ability to reduce iron. In some embodiments, the mutated microorganism has a capacity to reduce iron that is greater than about 2-times, greater than about 4-times, greater than about 6-times, greater than about 8-times, greater than about 10-times, or greater than about 40-times the capacity of the wild-type microorganism. In some embodiments, the mutated microorganism has a capacity to reduce iron that is greater than about 2-times, greater than about 4-times, greater than about 6-times, greater than about 8-times, or greater than about 10-times the capacity of the wild-type microorganism. In some embodiments, the mutation results in about a 2-fold, 4-fold, 6-fold, 8-fold, 10-fold, 40-fold increase in iron reduction capacity compared to the wild-type microorganism. In some embodiments, the mutation results in about a 2-fold, 4-fold, 6-fold, 8-fold, or 10-fold increase in iron reduction capacity compared to the wild-type microorganism

### Substrate

The processes disclosed herein are carried out in the presence of a substrate mixture, which can serve as an electron donor. Accordingly, in some embodiments, the substrate mixture of step (a) comprises a reducing agent. In some embodiments, the substitute mixture comprises waste products.

In some embodiments, the substrate mixture comprises carbonaceous waste products, elemental sulfur, substances containing partially reduced sulfur forms, or mixtures thereof.

In some embodiments, the substrate mixture comprises molasses, sugar, beet sugar, cane sugar, dextrose hydrolyzed from corn, dextrose hydrolyzed from cornstarch, sucrose containing materials, fermentation products thereof, or mixtures thereof. In some embodiments, the substrate is derived from yeast. In some embodiments, the substrate is not derived from yeast.

In some embodiments, the substrate mixture comprises acetate, lactate, glucose, or mixtures thereof. In some embodiments, the substrate mixture comprises acetate, lactate, or glucose derived from molasses, sugar, beet sugar, cane sugar, dextrose hydrolyzed from corn or dextrose hydrolyzed from cornstarch, sucrose containing materials or palm oil. In some embodiments, the substrate is acetate, lactate, or glucose. In some embodiments, the substrate is acetate or lactate. In some embodiments, the substrate is acetate, lactate, or glucose derived from palm oil.

### Oxide Ore

An oxide ore suitable for use in the disclosed processes is any oxide ore comprising one or more metals (e.g., nickel and/or cobalt) of commercial value or where removing one or more metals is of commercial value.

In some embodiments, the oxide ore is a laterite ore. In some embodiments, the oxide ore is a nickel laterite.

In some embodiments, the oxide ore is milled prior to step (a). In some embodiments, the milled oxide ore comprises particles less than about 25 mm diameter. In some embodiments, the milled oxide ore comprises particles less than about 10 mm diameter. In some embodiments, the milled oxide ore comprises particles less than about 5 mm diameter.

In some embodiments, the milled oxide ore comprises particles less than about 1 mm diameter.

In some embodiments, the milled ore comprises particles from about 1 mm to about 10 mm in diameter. In some embodiments, the milled ore comprises particles from about 1 mm to about 5 mm in diameter. In some embodiments, the milled ore comprises particles from about 1 mm to about 3 mm in diameter. In some embodiments, the diameter refers to the average diameter of the particle.

In some embodiments, the oxide ore is crushed prior to step (a). In some embodiments, the crushed oxide ore comprises particles less than about 25 mm in diameter. In some embodiments, the crushed oxide ore comprises particles less than about 10 mm in diameter. In some embodiments, the crushed oxide ore comprises particles less than about 6 mm in diameter. In some embodiments, the crushed oxide ore comprises particles less than about 4 mm in diameter. In some embodiments, the crushed oxide ore comprises particles less than about 2 mm in diameter. In some embodiments, the crushed ore comprises particles from about 2 mm to about 10 mm in diameter. In some embodiments, the crushed ore comprises particles from about 4 mm to about 8 mm in diameter. In some embodiments, the diameter refers to the average diameter of the particle.

In some embodiments, the oxide ore comprises from about 1% to about 90% by weight of one or more ferric iron minerals, e.g., about 1%, about 2.5%, about 5%, about 7.5%, about 10%, about 12.5%, about 15%, about 17.5%, about 20%, about 22.5%, about 25%, about 27.5%, about 30%, about 32.5%, about 35%, about 37.5%, about 40%, about 42.5%, about 45%, about 47.5%, about 50%, about 52.5%, about 55%, about 57.5%, about 60%, about 62.5%, about 65%, about 67.5%, about 70%, about 72.5%, about 75%, about 77.5%, about 80%, about 82.5%, about 85%, about 87.5%, or about 90%, including all ranges and subvalues therebetween. In some embodiments, the oxide ore comprises from about 1% to about 25% by weight of one or more ferric iron minerals. In some embodiments, the oxide ore comprises from about 1% to about 20% by weight iron. In some embodiments, the oxide ore comprises from about 5% to about 15% by weight iron.

In some embodiments, the oxide ore comprises from about 1% to about 90% by weight of one or more manganese minerals, e.g., about 1%, about 2.5%, about 5%, about 7.5%, about 10%, about 12.5%, about 15%, about 17.5%, about 20%, about 22.5%, about 25%, about 27.5%, about 30%, about 32.5%, about 35%, about 37.5%, about 40%, about 42.5%, about 45%, about 47.5%, about 50%, about 52.5%, about 55%, about 57.5%, about 60%, about 62.5%, about 65%, about 67.5%, about 70%, about 72.5%, about 75%, about 77.5%, about 80%, about 82.5%, about 85%, about 87.5%, or about 90%, including all ranges and subvalues therebetween. In some embodiments, the oxide ore comprises from about 1% to about 25% by weight of one or more manganese minerals. In some embodiments, the oxide ore comprises from about 1% to about 20% by weight manganese. In some embodiments, the oxide ore comprises from about 5% to about 15% by weight manganese.

In some embodiments, the oxide ore comprises about 0.1% to about 15% by weight nickel, e.g., about 0.1%, about 0.5%, about 0.75%, about 1%, about 2%, about 3%, about 4%, about 5%, about 6%, about 7%, about 8%, about 9%, about 10%, about 11%, about 12%, about 13%, about 14%, or about 15%, including all ranges and values therebetween.

In some embodiments, the oxide ore comprises about 0.1% to about 5% by weight nickel. In some embodiments, the oxide ore comprises about 0.1% to about 2.5% by weight nickel.

In some embodiments, the nickel in the oxide ore is associated with one or more ferric iron minerals, manganese minerals, quartz, and/or magnesium silicates. In some embodiments, the nickel in the oxide ore is associated with one or more ferric iron minerals. In some embodiments, the nickel in the oxide ore is associated with one or more manganese minerals.

In some embodiments, a majority (e.g., greater than 50% by weight, greater than 60% by weight, greater than 70% by weight, etc.) of the nickel in the oxide ore is associated with one or more ferric iron minerals. In some embodiments, substantially all of the nickel in the oxide ore is associated with one or more ferric iron minerals. In some embodiments, a majority of nickel in the oxide ore is associated with one or more ferric iron minerals. In some embodiments, the one or more ferric iron minerals is a ferric iron (oxy)hydroxide. In some embodiments, the one or more ferric iron minerals is α-FeO·OH (goethite). In some embodiments, about 10% to about 90% of the nickel in the oxide ore is associated with the goethite. In some embodiments, about 40% to about 70% of the nickel in the oxide ore is associated with the goethite. In some embodiments, about 50% to about 60% of the nickel in the oxide ore is associated with the goethite.

In some embodiments, the oxide ore of the present disclosure comprises about 0.01% to about 2.0% by weight cobalt, e.g., about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2%, including all ranges and values therebetween. In some embodiments, the oxide ore comprises about 0.1% to about 0.5% by weight cobalt.

In some embodiments, the cobalt is solubilized in the aqueous phase of step (b).

### Ligand

In some embodiments of the disclosed processes, the mixture of step (a) comprises (i) one or more microorganisms, (ii) the substrate mixture, (iii) the ore, (iv) a ligand, (v) an optional electron shuttle, and (vi) the aqueous phase. In some embodiments, the ligand added in step (a) stabilizes metals in solution.

In some embodiments of the disclosed processes, the mixture of step (a) comprises (i) one or more microorganisms, (ii) the substrate mixture, (iii) the ore, (iv) a ligand, and (v) the aqueous phase. In some embodiments, the ligand added in step (a) stabilizes metals in solution.

In some embodiments, the ligand is present in step (a) in an amount ranging from about 100 μM to 500 mM, e.g., 100 μM, 500 μM, 1 mM, 5 mM, 10 mM, 15 mM, 20 mM, 25 mM, 30 mM, 35 mM, 40 mM, 45 mM, 50 mM, 100 mM, 250 mM, or 500 mM including all ranges and values therebetween. In some embodiments, the ligand is present in step (a) in an amount ranging from about 100 μM to 50 mM, e.g., 100 μM, 500 μM, 1 mM, 5 mM, 10 mM, 15 mM, 20 mM, 25 mM, 30 mM, 35 mM, 40 mM, 45 mM, or 50 mM, including all ranges and values therebetween. In some embodiments, the ligand is present in an amount ranging from about 100 μM to 500 μM. In some embodiments, the ligand is present in an amount ranging from about 100 μM to 250 μM. In some embodiments, no more than 100 μM, 150 μM, 200 M, 250 μM, 300 μM, 350 μM, 400 μM, 450 μM, or 500 μM of ligand is present in the mixture of step (a).

In some embodiments, the ligand is an organic acid or combination of organic acids. In some embodiments, the ligand is an organic acid selected from the group consisting of malic acid, lactic acid, gluconic acid, pyruvic acid, succinic acid, ketoglutaric acid, oxalic acid, fumaric acid, citric acid, acetic acid, malonic acid, salicylic acid, acetohydroxamic acid, mugineic acid, benzoic acid, hydroxamic acid, and a combination thereof. In some embodiments, the ligand is an organic acid selected from the group consisting of malic acid, lactic acid, gluconic acid, pyruvic acid, succinic acid, ketoglutaric acid, oxalic acid, fumaric acid, citric acid, acetic acid, malonic acid, salicylic acid, acetohydroxamic acid, mugineic acid, benzoic acid, hydroxamic acid, and a combination thereof.

### Electron Shuttles

In some embodiments of the disclosed processes, the mixture of step (a) comprises (i) one or more microorganisms, (ii) the substrate mixture, (iii) the ore, (iv) an optional ligand, (v) an electron shuttle, and (v) the aqueous phase. In some embodiments, the electron shuttle added in step (a) promotes transfer of electrons to laterite minerals.

In some embodiments, the electron shuttle is present in step (a) in an amount ranging from about 1 μM to 250 μM, e.g., 1 μM, 5 μM, 10 μM, 50 μM, 100 μM, or 250 μM, including all ranges and values therebetween. In some embodiments, the ligand is present in an amount ranging from about 1 μM to 250 μM. In some embodiments, the ligand is present in an amount ranging from about 100 μM to 250 μM. In some embodiments, no more than 100 μM, 150 μM, 200 μM, 250 μM, 300 μM, 350 μM, 400 μM, 450 μM, or 500 μM of ligand is present in the mixture of step (a).

In some embodiments, the electron shuttle is present in step (a) in an amount ranging from about 1 g/L to 50 g/L, e.g., 1 g/L, 5 g/L, 10 g/L, 50 g/L, including all ranges and values therebetween. In some embodiments, the ligand is present in an amount ranging from about 1 g/L to 50 g/L. In some embodiments, the ligand is present in an amount ranging from about 100 g/L to 250 g/L. In some embodiments, no more than 1 g/L, 5 g/L, 10 g/L, or 50 g/L, of shuttle is present in the mixture of step (a).

In some embodiments, the electron shuttle is present in step (a) in a mass ratio to Fe ranging from about 0.1 to 5, including all ranges and values therebetween.

In some embodiments, the mixture of step (a) further comprises an electron shuttle. In some embodiments, the electron shuttle is an organic compound or combination of organic compounds. In some embodiments, the electron shuttle is an organic compound selected from the group consisting of flavins (including compounds that contain tricyclic heterocycle isoalloxazine or its isomer alloxazine, and derivatives thereof), quinones, humic acids, fulvic acids, biochar, and a combination thereof.

### Aqueous Phase

In some embodiments, the aqueous phase of step (a) comprises a buffer that maintains the pH of the solution or mixture within a certain range.

In some embodiments, the buffer maintains a pH of about 3 to 10. In some embodiments, the buffer maintains a pH of about 4-9. In some embodiments, buffer maintains a pH of about 5 to 9. In some embodiments, buffer maintains a pH of about 5 to 8. In some embodiments, the buffer maintains a pH of about 4. In some embodiments, the buffer maintains a pH of about 5. In some embodiments, the buffer maintains a pH of about 6. In some embodiments, the buffer maintains a pH of about 7. In some embodiments, the buffer maintains a pH of about 8.

### Process Parameters

In some embodiments, the processes disclosed herein are carried out as a continuous flow or semi-continuous flow process.

In some embodiments, the process is carried out by a heap leaching, column leaching, stirred tank leaching, or in situ process. In some embodiments, the in situ process comprises leaching metals from an oxide ore that has not been excavated.

### Step (a)

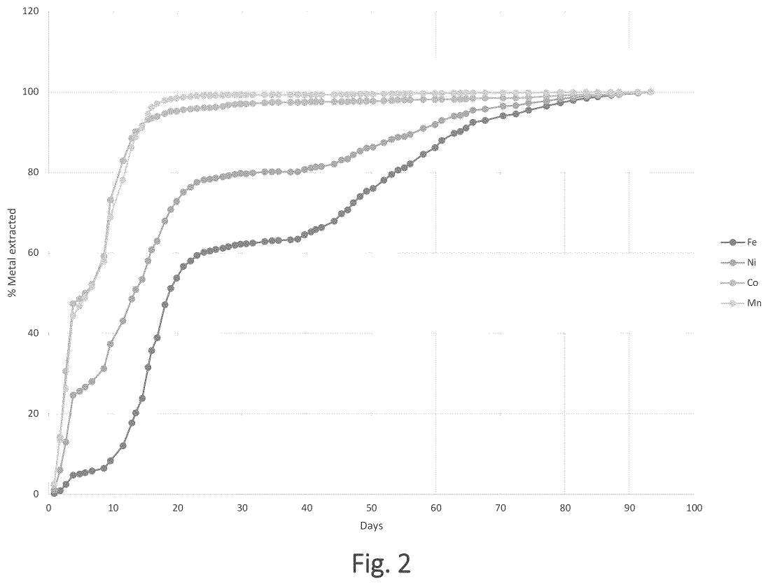

In some embodiments, the oxide ore is formed into a heap prior to step (a). In such cases, the processes disclosed herein can be characterized as heap leaching processes (see FIG. 2), where a crushed or milled ore is transported to a heap location and stacked, for example, onto an impervious liner or heap leach pad.

In some embodiments, the contacting or combining of step (a) comprises stirring or other form of agitation known in the art.

In some embodiments, the one or more microorganisms of step (a) are added to a slurry comprising substrate mixture, the ore, the optional ligand, and the aqueous phase.

In some embodiments, the one or more microorganisms are cultivated in growth medium in a bioreactor prior to step (a). In some embodiments, the one or more microorganisms of step (a) are derived from the ore.

### Step (b)

In some embodiments, the predetermined period of time of step (b) is from about 0.5 h to about 30 days, e.g., about 0.5 h, about 1 h, about 5 h, about 10 h, about 24 h, about 2 days, about 5 days, about 10 days, about 15 days, about 20 days, about 25 days, or about 30 days, including any range or value therebetween. In some embodiments, the predetermined period of time of step (b) is from about 1 h to about 5 d. In some embodiments, the predetermined period of time of step (b) is from about 1 h to about 2 d. In some embodiments, the predetermined period of time of step (b) is from about 1 h to about 24 h.

In some embodiments, the conditions of step (b) comprise maintaining the mixture at ambient temperature. In some embodiments, the conditions of step (b) comprise maintaining the mixture at a temperature from about 5° C. to about 100° C., e.g., about 5° C., about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., or about 100° C. including all ranges and values therebetween. In some embodiments, the conditions of step (b) comprise maintaining the mixture at a temperature from about 10° C. to about 50° C.

In some embodiments, the conditions of step (b) comprise maintaining the mixture at ambient pressure. In some embodiments, the conditions of step (b) comprise maintaining the mixture at an absolute pressure of about one atmosphere.

In some embodiments, the conditions of step (b) comprise maintaining the mixture at ambient temperature and pressure.

In some embodiments, the conditions of step (b) comprise maintaining the aqueous phase at a pH of from about 4-9. In some embodiments, the conditions of step (b) comprise maintaining the aqueous phase at a pH of from about 5-8. In some embodiments, the conditions of step (b) comprise maintaining the aqueous phase at a pH of from about 5-7. In some embodiments, the conditions of step (b) comprise maintaining the aqueous phase at a pH of from about 6-8. In some embodiments, the conditions of step (b) comprise maintaining the aqueous phase at a pH of from about 5-6.

In some embodiments, the conditions of step (b) comprise agitating the mixture. In some embodiments, the mixture is agitated by stirring.

In some embodiments, the one or more microorganisms used in the reduction of step (b) are grown under anaerobic or substantially anaerobic conditions. As described herein, the microorganisms can be cultivated in a bioreactor prior to introduction in step (a) or the microorganisms can be derived from the oxide ore, for example by providing appropriate nutrients and/or other media.

In some embodiments, the reduction of ferric iron to ferrous iron in step (b) is carried out under anoxic or substantially anoxic conditions.

In some embodiments, substantially all of the ferric iron in the ore is reduced to ferrous iron during step b). In some embodiments, at least 30%, at least 40%, at least 50%, at least 60%, greater than 70%, at least 80%, or at least 90%, by weight, of the ferric iron in the ore is reduced to ferrous iron during step b). In some embodiments, greater than 70%, greater than 80%, or greater than 90%, by weight, of the ferric iron in the ore is reduced to ferrous iron during step b).

In some embodiments, about 5% to about 99% by weight of the nickel in the ore is solubilized in the aqueous phase after step (b), e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%, including all ranges and values therebetween. In some embodiments, about 50% to about 80% by weight of the nickel in the ore is solubilized in the aqueous phase after step (b). In some embodiments, about 60% to about 75% by weight of the nickel in the ore is solubilized in the aqueous phase after step (b).

In some embodiments, about 1%, about 5%, about 10%, about 15%, about 20%, about 25%, about 30%, about 35%, about 40%, about 45%, about 50%. In some embodiments, about 1% to about 25% by weight of the nickel is solubilized per day from the ore. In some embodiments, about 1% to about 10% by weight of the nickel is solubilized per day from the ore. In some embodiments, about 3% to about 6% by weight of the nickel is solubilized per day from the ore.

In some embodiments, about 5% to about 90% by weight of the cobalt in the ore is solubilized in the aqueous phase after step (b). In some embodiments, at least about 30% by weight of the cobalt is solubilized in the aqueous phase after step (b). In some embodiments, about 30% to about 70% by weight of the cobalt in the ore is solubilized in the aqueous phase after step (b).

In some embodiments, about 1% to about 25% by weight of the cobalt is solubilized per day from the ore. In some embodiments, about 1% to about 10% by weight of the cobalt is solubilized per day from the ore. In some embodiments, about 2% to about 5% by weight of the cobalt is solubilized per day from the ore.

### Step (c)

In some embodiments, the leach solution of step (c) comprises dissolved amounts of ferrous iron, nickel, cobalt, and optionally, minor impurities. In some embodiments, the leach solution of step (c) comprises dissolved amounts of ferrous iron, nickel or cobalt, and optionally, minor impurities. In some embodiments, the leach solution of step (c) comprises dissolved amounts of ferrous iron, nickel, and optionally, minor impurities. In some embodiments, the leach solution of step (c) comprises dissolved amounts of ferrous iron, cobalt, and optionally, minor impurities. In some embodiments, the leach solution of step (c) comprises ferrous iron and an amount of dissolved nickel. In some embodiments, the leach solution of step (c) comprises ferrous iron and an amount of dissolved cobalt. In some embodiments, the leach solution of step (c) further comprises an amount of dissolved copper.

In some embodiments, the leach solution of step (c) further comprises an amount of other dissolved metals, such as chromium and manganese. In some embodiments, the dissolved nickel is in the form Ni²⁺ ions, hydrolyzed forms, and/or ligand-bound forms. In some embodiments, the dissolved cobalt is in the form of Co²⁺ ions, hydrolyzed forms, and/or ligand bound forms.

In some embodiments, the aqueous phase of step (c) is isolated as a pregnant leach solution from the mixture by sedimentation of the undissolved solids and decantation of the leach solution. In some embodiments, the aqueous phase of step (c) is isolated as a pregnant leach solution from the mixture from the mixture by gravity filtration. In some embodiments, the one or more metals in the pregnant leach solution of step (c) is nickel and/or cobalt. In some embodiments, the one or more metals is nickel. In some embodiments, the one or more metals in cobalt. In some embodiments, pregnant leach solution further comprises one or more dissolved metals, e.g., chromium or manganese, depending on the composition of the oxide ore undergoing processing.

In some embodiments, the pregnant leach solution of step (c) is substantially free of undissolved solids. In some embodiments, the pregnant leach solution of step (c) comprises small amounts of undissolved solids.

In some embodiments, the pregnant leach solution comprises more than 1000 ppm, more than 500 ppm, more than 250 ppm, more than 100 ppm, more than 50 ppm, more than 25 ppm, more than 10 ppm, more than 5 ppm, more than 1 ppm, or more than 0.1 ppm of the metal (e.g., nickel and/or cobalt) targeted for recovery.

In some embodiments, the remaining part of the mixture, following isolation of the leach solution is step (c), can be recycled. In some embodiments, the recycled mixture can be used as the aqueous phase of step (a).

Recovery of Dissolved Metals from Solution

The one or more metals dissolved in the leach solution of step (c) can be recovered according to the process described below or by variations thereof, as would be understood by one of skill in the art.

Accordingly, in some embodiments, the processes of the present disclosure further comprise: (d) contacting the pregnant leach solution of step (c) with a conjugate comprising a peptide and a polystyrene (PS) bead for a period of time under conditions that form a conjugate-metal ion complex, wherein the peptide is configured to selectively bind nickel and/or cobalt; (e) isolating the conjugate-metal ion complex of step (d); (f) eluting the metal ion(s) from the conjugate-metal ion complex by washing the complex with acid, thereby forming a solution; (g) transferring the solution of step (f) comprising the metal ions to an electrowinning circuit; (h) applying an electric current through the solution of step (f) comprising the metal ions; (i) collecting the resulting metal(s) on a surface of a cathode; and (j) recovering the metal(s) from the cathode.

In some embodiments, if a small amount of one or more additional divalent metals is present, prior to step (d), the divalent metal can be removed by contacting the solution of step (c) with an ion exchange resin selective for absorbing the metal.

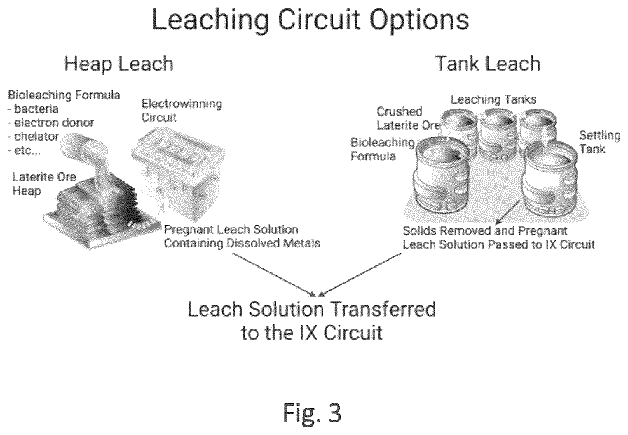

In some embodiments, the leach solution of step (d) comprises a conjugate-Ni^{II} ion coordination complex. In some embodiments, the conjugate-Ni^{II} ion coordination complex has the structure:

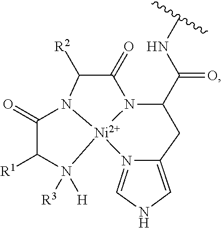

wherein R¹, R², and R³ are as defined herein. In some embodiments, the conjugate-Ni^{II} ion coordination complex has the structure provided in FIG. 3B, wherein R¹ and R² are as defined herein.

In some embodiments, greater than about 20%, greater than about 30%, greater than about 40%, greater than about 50%, greater than about 60%, greater than about 70%, greater than about 80%, greater than about 90%, or greater than about 95%, of the nickel in the leach solution is complexed to the conjugate after the period of time in step (d). In some embodiments, greater than about 50% of the nickel in the leach solution is complexed to the conjugate after the period of time in step (d). In some embodiments, greater than about 80% of the nickel in the leach solution is complexed to the conjugate after the period of time in step (d).

In some embodiments, the leach solution of step (d) comprises a conjugate-Co^{II} ion coordination complex. In some embodiments, the conjugate-Co^{II} ion coordination complex has the structure:

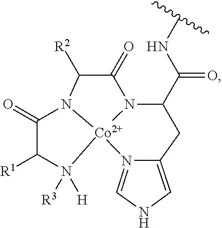

wherein R¹, R², and R³ are as defined herein.

In some embodiments, greater than about 20%, greater than about 30%, greater than about 40%, greater than about 50%, greater than about 60%, greater than about 70%, greater than about 80%, greater than about 90%, or greater than about 95%, of the cobalt in the leach solution is complexed to the conjugate after the period of time in step (d). In some embodiments, greater than about 50% of the cobalt in the leach solution is complexed to the conjugate after the period of time in step (d). In some embodiments, greater than about 80% of the cobalt in the leach solution is complexed to the conjugate after the period of time in step (d).

In some embodiments, the contacting of step (d) comprises the application of heat and/or stirring.

In some embodiments, the period of time in step (d) is from about 1 h to about 24 h, e.g., about 1 h, about 2 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 21 h, about 22 h, about 23 h, or about 24 h, including all ranges and values therebetween.

Isolating the conjugate-metal complex can be carried out by any means known in the art. In some embodiments, step (e) isolating the conjugate-metal ion complex comprises filtering off the leach solution.

In some embodiments, the acid of step (f) is a dilute acid. In some embodiments, the acid of step (f) is a weak acid. In some embodiments, the acid of step (f) has a pH ranging from about 2-5. In some embodiments, the acid of step (f) has a pH ranging from about 2-4.

In some embodiments, the solution of step (f) comprises greater than 20%, greater than 30%, greater than 40%, greater than 50%, greater than 60%, greater than 70%, greater than 80%, or greater than 90% of the nickel in the leach solution of step (a). In some embodiments, the solution of step (f) comprises greater than 50% of the nickel in the leach solution of step (a). In some embodiments, the solution of step (f) comprises greater than 60% of the nickel in the leach solution of step (a). In some embodiments, the solution of step (f) comprises greater than 70% of the nickel in the leach solution of step (a). In some embodiments, the solution of step (f) comprises greater than 80% of the nickel in the leach solution of step (a). In some embodiments, the solution of step (f) comprises greater than 90% of the nickel in the leach solution of step (a).

In some embodiments, the nickel recovered from the cathode in step (j) represents greater than about 30%, greater than about 40%, greater than about 50%, greater than about 60%, greater than about 70%, greater than about 80%, or greater than about 90% by weight of the nickel in the oxide ore. In some embodiments, the nickel recovered from the cathode in step (j) represents greater than about 50% by weight of the nickel in the oxide ore. In some embodiments, the nickel recovered from the cathode in step (j) represents greater than about 75% by weight of the nickel in the oxide ore. In some embodiments, the nickel recovered from the cathode in step (j) represents greater than about 90% by weight of the nickel in the oxide ore.

In some embodiments, the solution of step (f) comprises greater than 20%, greater than 30%, greater than 40%, greater than 50%, greater than 60%, greater than 70%, greater than 80%, or greater than 90% of the cobalt in the leach solution of step (a). In some embodiments, the solution of step (f) comprises greater than 50% of the cobalt in the leach solution of step (a). In some embodiments, the solution of step (f) comprises greater than 60% of the cobalt in the leach solution of step (a). In some embodiments, the solution of step (f) comprises greater than 70% of the cobalt in the leach solution of step (a). In some embodiments, the solution of step (f) comprises greater than 80% of the cobalt in the leach solution of step (a). In some embodiments, the solution of step (f) comprises greater than 90% of the cobalt in the leach solution of step (a).

In some embodiments, the cobalt recovered from the cathode in step (j) represents greater than about 30%, greater than about 40%, greater than about 50%, greater than about 60%, greater than about 70%, greater than about 80%, or greater than about 90% by weight of the cobalt in the oxide ore. In some embodiments, the cobalt recovered from the cathode in step (j) represents greater than about 50% by weight of the cobalt in the oxide ore. In some embodiments, the cobalt recovered from the cathode in step (j) represents greater than about 75% by weight of the cobalt in the oxide ore.

In some embodiments, nickel and cobalt are recovered together in step (j), and the mixture is further processed to recover each metal individually. In some nickel and cobalt are recovered separately in step (j), for example when a conjugate selective for nickel and a conjugate selective for cobalt are used in the process.

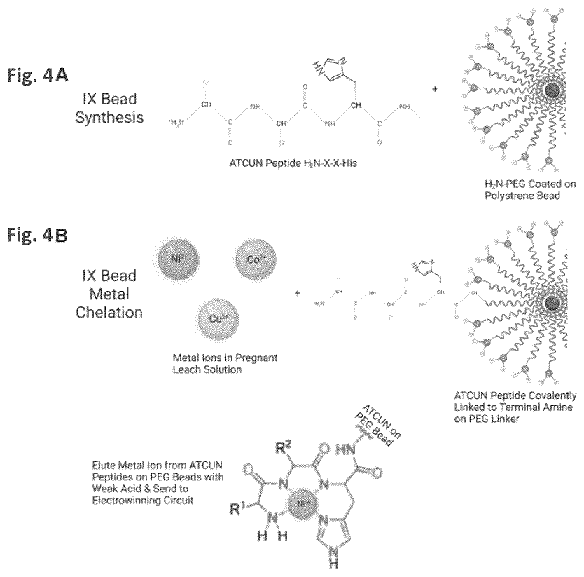

In some embodiments, the one or more metals is recovered according to a process provided in FIG. 4A, FIG. 4B, FIG. 4C, FIG. 4D, FIG. 4E, or FIG. 4F.

### Conjugates

The present disclosure provides conjugates comprising a peptide that can be configured to selectively coordinate one or more metal ions, e.g., Ni^{II} and/or Co^{II}, dissolved in an aqueous solution. In some embodiments, the conjugates comprise a peptide attached to a polystyrene (PS) bead. In some embodiments, the peptide is covalently attached to the bead.

In some embodiments, the conjugates of the present disclosure comprise the structure:

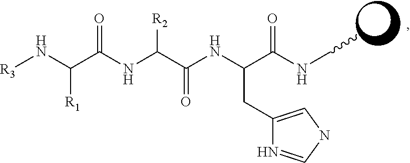

wherein R₁ and R₂ are each independently H or a side chain of an amino acid residue (as disclosed herein), R₃ is H or a capping group (e.g., —C(O)—, S(O)₂—, and the like), [symbol] represents a polyethylene glycol (PEG) group, and [symbol] represents a PS bead.

In some embodiments, the conjugates of the present disclosure are prepared as described in FIG. 3A.

### Selectivity

In some embodiments, the conjugate is configured to selectively bind nickel. In some embodiments, the conjugate has a selectivity for nickel over the other metals present in the ore (e.g., iron) that is greater than 2:1, greater than 3:1, greater than 4:1, greater that 5:1, greater than 6:1, greater than 8:1, greater than 10:1, greater than 12:1, or greater than 15:1. In some embodiments, the conjugate has a selectivity for nickel over the other metals present in the ore that is greater than 3:1. In some embodiments, the conjugate has a selectivity for nickel over iron that is greater than 3:1. In some embodiments, the conjugate has a selectivity for nickel over iron that is greater than 5:1. In some embodiments, the conjugate has a selectivity for nickel over iron that is greater than 10:1. In some embodiments, the nickel is in the form of Ni^{II} ions. In some embodiments, the conjugate has a binding affinity (Kd) for Ni^{II} of from about 1×10⁻¹⁶ to about 1×10⁻⁸ M⁻¹.

In some embodiments, the conjugate is configured to selectively bind cobalt. In some embodiments, the conjugate has a selectivity for cobalt over the other metals present in the ore that is greater than 3:1. In some embodiments, the conjugate has a selectivity for cobalt over iron that is greater than 3:1. In some embodiments, the conjugate has a selectivity for cobalt over iron that is greater than 5:1. In some embodiments, the conjugate has a selectivity for cobalt over iron that is greater than 10:1. In some embodiments, the cobalt is in the form of Co^{II} ions. In some embodiments, the conjugate has a binding affinity (Kd) for Co^{II} of from about 1×10⁻¹⁶ to about 1×10⁻⁸ M⁻¹.

In some embodiments, the conjugate has a selectivity for nickel over cobalt that is greater than 3:1. In some embodiments, the conjugate has a selectivity for nickel over cobalt that is greater than 5:1. In some embodiments, the conjugate has a selectivity for nickel over cobalt that is greater than 10:1.

In some embodiments, the conjugate has a selectivity for cobalt over nickel that is greater than 3:1. In some embodiments, the conjugate has a selectivity for cobalt over nickel that is greater than 5:1. In some embodiments, the conjugate has a selectivity cobalt over nickel that is greater than 10:1.

### Peptides

In some embodiments, the peptide comprises from 3 to 30 amino acid residues. In some embodiments, the peptide comprises from 3 to 20 amino acid residues. In some embodiments, the peptide comprises from 3 to 10 amino acid residues. In some embodiments, the peptide comprises from 3, 4, or 5 amino acid residues. In some embodiments, the peptide comprises 3 amino acid residues. In some embodiments, the peptide comprises amino acid residues derived from natural amino acids. In some embodiments, the peptide comprises amino acid residues derived from unnatural amino acids. In some embodiments, the peptide comprises only amino acids derived from natural amino acids. In some embodiments, the peptide comprises a histidine residue.

In some embodiments, each amino acid residue is independently selected from the group consisting of a glycine, alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, and a-hydroxymethylserine residue.

In some embodiments, the peptide comprises an amino acid sequence found in SCO₄₂₂₆, RcnR, NmtR, HypA, or E coli HypB. In some embodiments, the sequence is effective for binding nickel ions.

In some embodiments, the peptide comprises one of the following amino acid sequences:

MAHFMDVHRGMHGITSDQLHQAHQADLAVEKDENVHFEQAWADPASGTIY
CLSEGPSAEAVQRVHERAGHKADEIHEVPLSA
(SCO4226 protein; SEQ ID 1);
Hexa-His (His6; SEQ ID 2);
(Glu-Cys)20Gly (E. coli sp.; SEQ ID 3);
Ac-KGCHLDAQMIADAAPRLPLDDNGILFIENVGNLVCP-NH₂
(E. coli HypB; SEQ ID 4);
NH₂-MCTTCGCGEG-NH₂ (E. coli HypB; SEQ ID 5).

In some embodiments, the conjugate comprises a peptide having the structure: —NH-X_{AA1}-X_{AA2}-His-, wherein X_{AA1} and X_{AA2} are each independently an amino acid residue other than histidine.

In some embodiments, the conjugate comprises a peptide having the structure: —NH-X_{AA1}-X_{AA2}-His-X_{AA3}, wherein X_{AA1}, X_{AA2}, and X_{AA3} are each independently an amino acid residue other than histidine.

In some embodiments, each X_{AA1} and X_{AA2} is independently a residue of glycine, alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or a-hydroxymethylserine.

In some embodiments, —NH-X_{AA1}-X_{AA2}-His- is selected from the group consisting of: -HN-DAH, -HN-DTH, -HN-VIH, -HN-MDH, -HN-RFH, -HN-RTH, -HN-HSH, -HN-GGH, -HN-GKH, -HN-KGH, -HN-KKH, -HN-YYH, -HN-MNH, -HN-kGH, -HN-kGH, -HN-GkH, and -HN-kkH.

In some embodiments, X_{A}m is an amino acid residue comprising a side chain. In some embodiments, X_{AA3} is an amino acid residue comprising a side chain that is positively charged.

In some embodiments, the side chain comprises an amine or guanidine moiety. In some embodiments, X_{AA3} is a lysine, ornithine, arginine, or homoarginine residue. In some embodiments, X_{AA3} is a lysine residue. In some embodiments, X_{AA3} is an arginine residue.

In some embodiments, the conjugate comprises a peptide having the structure:

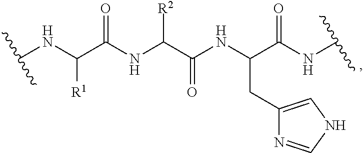

wherein R¹ and R² are each independently H or a side chain of an amino acid. In some embodiments, the side chain is positively charged. In some embodiments, the side chain comprises an amine or guanidine moiety.

In some embodiments, the conjugate comprises a peptide having the structure:

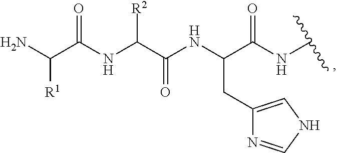

wherein R¹ and R² are each independently H or a side chain of an amino acid. In some embodiments, the side chain is positively charged. In some embodiments, the side chain comprises an amine or guanidine moiety.

In some embodiments, R¹ and R² are each independently the side of a glycine, alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or a-hydroxymethylserine residue. In some embodiments, R¹ and R² are each independently the side chain of a lysine, ornithine, arginine, or homoarginine residue.

The peptides disclosed herein can be configured to selectively bind nickel, cobalt, or other metal of interest, e.g., by modifying the metal binding motif and/or the properties of the peripheral amino acids (i.e., the amino acids outside the binding motif) that may be included in the conjugate. The desired selectivity may depend on the nature of the ore, the process being implemented to extract one or more metals from an ore, and/or other considerations appreciated by one of skill in the art.

In some embodiments, the peptide is configured to selectively bind nickel. It would be appreciated by those skilled in the art that the selectivity for a particular metal, e.g., nickel, could be attenuated by modifying the structure of the peptide, such as the —NH-X_{AA1}-X_{AA2}-His-defined herein. In some embodiments, the peptide has a selectivity for nickel over the other metals present in the ore that is greater than 3:1. In some embodiments, the peptide has a selectivity for nickel over iron that is greater than 3:1. In some embodiments, the peptide has a selectivity for nickel over iron that is greater than 5:1. In some embodiments, the peptide has a selectivity for nickel over iron that is greater than 10:1. In some embodiments, the nickel is in the form of Ni^{II} ions. In some embodiments, the peptide has a binding affinity (Kd) for Ni^{II} of from about 1×10⁻¹⁶ to about 1×10⁻⁸ M⁻¹.

In some embodiments, the peptide is configured to selectively bind cobalt. It would be appreciated by those skilled in the art that the selectivity for a particular metal, e.g., cobalt, could be attenuated by modifying the structure of the peptide, such as the —NH-X_{AA1}-X_{AA2}-His-defined herein. In some embodiments, the peptide has a selectivity for cobalt over the other metals present in the ore that is greater than 3:1. In some embodiments, the peptide has a selectivity for cobalt over iron that is greater than 3:1. In some embodiments, the peptide has a selectivity for cobalt over iron that is greater than 5:1. In some embodiments, the peptide has a selectivity for cobalt over iron that is greater than 10:1. In some embodiments, the cobalt is in the form of Co^{II} ions. In some embodiments, the peptide has a binding affinity (Kd) for Co^{II} of from about 1×10⁻¹⁶ to about 1×10⁻⁸ M⁻¹.

In some embodiments, the peptide has a selectivity for nickel over cobalt that is greater than 3:1. In some embodiments, the peptide has a selectivity for nickel over cobalt that is greater than 5:1. In some embodiments, the peptide has a selectivity for nickel over cobalt that is greater than 10:1.

In some embodiments, the peptide has a selectivity for cobalt over nickel that is greater than 3:1. In some embodiments, the peptide has a selectivity for cobalt over nickel that is greater than 5:1. In some embodiments, the peptide has a selectivity cobalt over nickel that is greater than 10:1.

### Polystyrene (PS) Beads

The PS beads disclosed herein can include any properties suitable for use in metal recovery applications. In some embodiments, the PS beads are ion exchange beads. In some embodiments, the PS beads comprise crosslinked polystyrene. In some embodiments, the PS beads comprise polystyrene crosslinked with divinylbenzene. In some embodiments, the PS beads are porous polystyrene beads (PPB). The beads for use in the disclosed applications are stable under the conditions implemented.

In some embodiments, the PS beads have a bead size ranging from about 100 μm to about 1500 μm, e.g., about 100 μm, about 200 μm, about 300 μm, about 400 μm, about 500 μm, about 600 μm, about 700 μm, about 800 μm, about 900 μm, about 1000 μm, about 1100 μm, about 1200 μm, about 1300 μm, about 1400 μm, or about 1500 μm, including all ranges and values therebetween.

In some embodiments, the PS beads have a pore radius ranging from about 100 Å to about 1000 Å, e.g., about 100 Å, 200 Å, 300 Å, 400 Å, 500 Å, 600 Å, 700 Å, 800 Å, 900 Å, or 1000 Å, including all ranges and values therebetween. In some embodiments, the PS beads have a pore radius ranging from about 300 Å to about 700 Å.

In some embodiments, the PS beads have a total porosity greater than about 30%, greater than about 40%, greater than about 50%, greater than about 70%, greater than about 80%, or greater than about 90%. In some embodiments, the PS beads have a total porosity ranging from about 30% to about 90%. In some embodiments, the PS beads have a total porosity ranging from about 30% to about 60%.

In some embodiments, the PS bead comprises a plurality of H₂N-PEG groups. In some embodiments, the H₂N-PEG groups are coated on the PS bead. In some embodiments, the H₂N-PEG groups are on the surface of the PS bead.

In some embodiments, the conjugate comprises a peptide that is covalently linked to the PS bead. In some embodiments, one or more peptides are covalently linked to the PS bead.

In some embodiments, the peptide is covalently linked to the PS bead by an amine of the PEG group, as shown above.

### Numbered Embodiments

1. A leaching process for recovering nickel and/or cobalt from an oxide ore, the process comprising:
   (a) combining (i) one or more microorganisms, (ii) a substrate mixture, (iii) the oxide ore, (iv) an optional ligand, and (v) an aqueous phase to form a mixture;
   (b) maintaining the mixture of step a) for a pre-determined period of time under conditions sufficient to at least partially reduce an amount of ferric iron in the ore to ferrous iron, wherein the ferrous iron and nickel are solubilized in the aqueous phase; and
   (c) isolating the aqueous phase as a leach solution comprising the ferrous iron and an amount of dissolved nickel, from the mixture.
2. The process of embodiment 1, wherein the one or more microorganisms of step (a) are added to a slurry comprising substrate mixture, the ore, the optional ligand, and the aqueous phase.
3. The process of embodiment 1 or 2, wherein the one or more microorganisms of step (a) are derived from the ore.
4. The process of any one of embodiments 1-3, wherein the one or more microorganisms produce an enzyme or enzyme system selected from the group consisting of CymA, DFE, DmkA/DmkB, EetA/EetB, FmnA/FmnB, GACE, MtrA/MtrA/MtrC, Ndh2, OmcF/OmcS/OmcZ, PplA, and T4ap, or combinations thereof.
5. The process of any one of embodiments 1-4, wherein the one or more microorganisms are selected from the group consisting of Thermincola potens JR (NC_014152.1), Listeria monocytogenes (NC_003210.1), Clostridium celerecrescens (NZ_PGET01000001.1), Bacillus infernus, Bacillus subterraneus, Bacillus pseudormus MC02, Bacillus subterraneus, Thermoanaerobacter siderophilus (NZ_CM001486.1), Carboxydothermus ferrireducens (NZ_ATYG01000001.1), Carboxydothermus siderophilus, Carboxydothermus pertinax (NZ_BDJK01000055.1), Carboxydocella thermautotrophica (NZ_CP028491.1), Moorella humiferrea (NZ_PVXM01000006.1), Geosporobacter ferrireducens IRF9, Thermotalea metallivorans B2-1(T), Pelosinus fermentans, Thermotoga maritima (NC_023151.1), Sinirhodobacter ferrireducens (NZ_SAVB01000001.1), Acidiphilium SJH, Acidiphilium PK40, Acidiphilium PK46, Acidiphilium KPWI4, Acidiphilium cryptum JF-5, Acidocella facilis, Acidocella M21, Acidocella PFBC, Acidocella PWB4, Shewanella putrefaciens (NZ_CP066370.1), Shewanella oneidensis MR-1(NC_004347.2), Shewanella alga strain BrY (NZ_CP046378.1), Shewanella amazonensis sp. nov., Shewanella putrefaciens IR-1, Shewanella sp. HN-41, Shewanella putrefaciens CN-32, Shewanella putrefaciens 200R, Shewanella oneidensis MR-1 Shewanella baltica W3-6-1, Shewanella sp.PV-4, Shewanella peizotolerans WP3, Shewanella decolorationis, Shewanella frigidimarina, Shewanella gelidimarina, Shewanella loihica, Shewanella pealeana, Serratia plymuthica (NC_015567.1), Serratia fonticola (NZ_CP011254.1), Aeromonas hydrophila (NZ_CP050851.1), Klebsiella oxytoca (NZ_CP033844.1), Ferrimonas balearica (NC_014541.1), Frateuria-like isolate WJ2, Desulfovibrio ferrophilus (NZ_AP017378.1), Desulfuromusa ferrireducens, Desulfiuromonas svalbardensis, Desulfiuromonas acetoxidans (JABWTG01), Geobacter sulfurreducens (NC_002939.5), Geobacter bemidjiensis(NC_011146.1), Geobacter metallireducens (NC_007517.1), Geobacter pelophilus strain Dfr2 (NZ_JAHCVJ010000001.1), Geobacter daltonii (NC_011979.1), Geobacter chapellei (NZ_JAHDYS010000001.1), Geobacter psychrophilus sp. nov, Geobacter bremensis sp. nov., Geobacter lovleyi sp. nov. strain SZ, Geobacter luticola, Geobacter pickeringii, Geobacter argillaceus, Geobacter uraniireducens, Desulfosediminicola ganghwensis (NZ_CP050699.1), Desulfosediminicola flagellatus (NZ_CP050698.1), Anaeromyxobacter dehalogenans (NC_011891.1), Anaeromyxobacter Strain FAc12, Geoalkalibacter subterraneus (NZ_CP010311.1), Geoalkalibacter ferrihydriticus (NZ_FNGU01000001.1), Rhodoferax ferrireducens (NC_007908.1), Ferribacterium limneticum (NZ_CP075189.1), Geothrix fermentans (NZ_KE386810.1), Geovibrio ferrireducens, Deferribacter thermophilus, Pyrobaculum islandicum (NC_008701.1), Pyrodictium abyssi, Methanopyrus kandleri (NC_003551.1), Archaeoglobus fulgidus (NZ_CP006577.1), Pyrococcus furiosus (NZ_CP023154.1), Methanococcus thermolithotrophicus (NZ_AQXV01000055.1), Ferroglobus placidus (NC_013849.1), Geoglobus acetivorans sp. nov., Acidobacterium capsulatum, Acidobacterium PK35, Acidobacterium RIT23, Acidobacterium WJ7, Telmatospirillum (Alphaproteobacterial Genus) (PRJNA561022), Sideroxydans lithotrophicus ES-1, Magnetospirillum magneticum AMB-1, Candidatus Tectomicrobia, Candidatus Zixibacteria, Candidatus Tectomicrobia, Candidatus Dadabacteria, Candidatus Handelsmanbacteria, Carboxydothermus hydrogenoformans, Carboxydothermus islandicus, Telmatospirillum siberiense, Nitrosococcus halophilus, Skermanella stibiiresistens, Insolitispirillum peregrinum, Magnetospirillum magneticum, and mixtures thereof.
6. The process of any one of embodiments 1-5, wherein the one of more microorganisms is a Shewanella spp.
7. The process of any one of embodiments 1-6, wherein the one or more microorganisms is Shewanella putrefaciens IR-1, Shewanella putrefaciens CN-32, or Shewanella putrefaciens 200R
8. The process of any one of embodiments 1-7, wherein the one or more microorganisms is Shewanella putrefaciens 200R.
9. The process of any one of embodiments 1-8, wherein the one or more microorganisms is Shewanella putrefaciens IR-1.
10. The process of any one of embodiments 1-6, wherein the one or more microorganisms is Shewanella putrefaciens CN-32.
11. The process of any one of embodiments 1-10, wherein the microorganism possesses one or more mutations.
12. The process of any one of embodiments 1-11, wherein the microorganism is mutated to enhance its ability to reduce iron.
13. The process of any one of embodiments 1-12, wherein the substrate mixture comprises a reducing agent.
14. The process of any one of embodiments 1-13, wherein the substrate mixture comprises carbonaceous waste products, elemental sulfur, substances containing partially reduced sulfur forms, or mixtures thereof.
15. The process of any one of embodiments 1-13, wherein the substrate mixture comprises molasses, sugar, beet sugar, cane sugar, dextrose hydrolyzed from corn, dextrose hydrolyzed from cornstarch, sucrose containing materials, fermentation products thereof, or mixtures thereof.
16. The process of any one of embodiments 1-15, wherein the substrate is an acetate, lactate, or glucose derived from palm oil, molasses, sugar, beet sugar, cane sugar, dextrose hydrolyzed from corn, dextrose hydrolyzed from cornstarch, or sucrose containing materials.
17. The process of any one of embodiments 1-16, wherein the oxide ore is a laterite ore.
18. The process of any one of embodiments 1-17, wherein the oxide ore is milled.
19. The process of embodiment 18, wherein the milled oxide ore comprises particles less than about 1 mm in diameter.
20. The process of any one of embodiments 1-17, wherein the oxide ore is crushed.
21. The process of embodiment 20, wherein the crushed oxide ore comprises particles less than about 6 mm in diameter.
22. The process of any one of embodiments 1-21, wherein the oxide ore comprises from about 1% to about 25% by weight of one or more ferric iron minerals.
23. The process of any one of embodiments 1-22, wherein the oxide ore comprises from about 5% to about 15% by weight iron.
24. The process of any one of embodiments 1-23, wherein the oxide ore comprises about 0.1% to about 15% by weight nickel.
25. The process of any one of embodiments 1-24, wherein the oxide ore comprises about 0.1% to about 5% by weight nickel.
26. The process of any one of embodiments 1-25, wherein the oxide ore comprises about 0.1% to about 2.5% by weight nickel.
27. The process of any one of embodiments 1-26, wherein the nickel in the oxide ore is associated with one or more ferric iron minerals, quartz, and magnesium silicates.
28. The process of any one of embodiments 1-27, wherein a majority of nickel in the oxide ore is associated with one or more ferric iron minerals.
29. The process of embodiment 27 or 28, wherein the one or more ferric iron minerals is ferric iron (oxy)hydroxides or α-FeO·OH (goethite).
30. The process of embodiment 29, wherein about 40% to about 70% of the nickel in the oxide ore is associated with the goethite
31. The process of embodiment 29 or 30, wherein about 50% to about 60% of the nickel in the oxide ore is associated with the goethite
32. The process of any one of embodiments 1-33, wherein the oxide ore further comprises cobalt.
33. The process of any one of embodiments 1-32, wherein the oxide ore comprises about 0.01% to about 2.0% by weight cobalt.
34. The process of any one of embodiments 1-33, wherein the oxide ore comprises about 0.1% to about 0.5% by weight cobalt.
35. The process of any one of embodiments 1-34, wherein the cobalt is solubilized in the aqueous phase of step (b).
36. The process of any one of embodiments 1-35, wherein the mixture of step (a) comprises (i) one or more microorganisms, (ii) the substrate mixture, (iii) the ore, (iv) a ligand, and (v) the aqueous phase.
37. The process of any one of embodiments 1-36, wherein the ligand is an organic acid.
38. The process of any one of embodiments 1-37, wherein the ligand is a combination of organic acids.
39. The process of any one of embodiments 1-37, wherein the ligand is an organic acid selected from the group consisting of malic acid, lactic acid, gluconic acid, pyruvic acid, succinic acid, ketoglutaric acid, oxalic acid, fumaric acid and citric acid, or a combination thereof.
40. The process of any one of embodiments 1-39, wherein the aqueous phase of step (a) comprises a buffer.
41. The process of embodiment 40, wherein the buffer maintains a pH of about 5 to 8.
42. The process of any one of embodiments 1-41, wherein the predetermined period of time of step (b) is from about 1 h to about 30 days.
43. The process of any one of embodiments 1-42, wherein the predetermined period of time of step (b) is from about 1 h to about 24 h.
44. The process of any one of embodiments 1-43, wherein the conditions of step (b) comprise maintaining the mixture at a temperature from about 5° C. to about 75° C.
45. The process of any one of embodiments 1-44, wherein the conditions of step (b) comprise maintaining the mixture at a temperature from about 10° C. to about 50° C.
46. The process of any one of embodiments 1-45, wherein the conditions of step (b) comprise maintaining the mixture at an absolute pressure of about one atmosphere.
47. The process of any one of embodiments 1-46, wherein the conditions of step (b) comprise maintaining the aqueous phase at a pH of from about 5-8.
48. The process of any one of embodiments 1-47, wherein the conditions of step (b) comprise agitating the mixture.
49. The process of embodiment 48, wherein the mixture is agitated by stirring.
50. The process of any one of embodiments 1-49, wherein the one or more microorganisms used in the reduction of step (b) are grown under anaerobic or substantially anaerobic conditions.
51. The process of any one of embodiments 1-50, wherein the reduction of ferric iron to ferrous iron is carried out under anoxic or substantially anoxic conditions.
52. The process of any one of embodiments 1-51, wherein substantially all of the ferric iron in the ore is reduced to ferrous iron during step b).
53. The process of any one of embodiments 1-52, wherein at least 30%, at least 40%, at least 50%, at least 60%, greater than 70%, at least 80%, or at least 90%, by weight, of the ferric iron in the ore is reduced to ferrous iron during step b).
54. The process of any one of embodiments 1-53, wherein greater than 70%, greater than 80%, or greater than 90%, by weight, of the ferric iron in the ore is reduced to ferrous iron during step b).
55. The process of any one of embodiments 1-54, wherein the process is carried out as a continuous flow or semi-continuous flow process.
56. The process of any one of embodiments 1-55, wherein the process is carried out as a heap leaching, column leaching, stirred tank leaching, or in situ process.
57. The process of any one of embodiments 1-56, wherein about 5% to about 90% by weight of the nickel in the ore is solubilized in the aqueous phase after step (b).
58. The process of any one of embodiments 1-57, wherein about 50% to about 80% by weight of the nickel in the ore is solubilized in the aqueous phase after step (b).
59. The process of any one of embodiments 1-58, wherein about 1% to about 10% by weight of the nickel is solubilized per day from the ore.
60. The process of any one of embodiments 1-59, wherein about 3% to about 6% by weight of the nickel is solubilized per day from the ore.
61. The process of any one of embodiments 32-60, wherein about 5% to about 90% by weight of the cobalt in the ore is solubilized in the aqueous phase after step (b).
62. The process of any one of embodiments 32-61, wherein at least about 30% by weight of the cobalt is solubilized in the aqueous phase after step (b).
63. The process of any one of embodiments 32-62, wherein about 30% to about 70% by weight of the cobalt in the ore is solubilized in the aqueous phase after step (b).
64. The process of any one of embodiments 32-63, wherein about 1% to about 10% by weight of the cobalt is solubilized per day from the ore.
65. The process of any one of embodiments 32-64, wherein about 2% to about 5% by weight of the cobalt is solubilized per day from the ore.
66. The process of any one or embodiments 32-65, wherein the leach solution of step (c) further comprises an amount of dissolved cobalt.
67. The process of any one of embodiments 1-66, wherein the aqueous phase of step (c) is isolated from the mixture by sedimentation of the undissolved solids and decantation of the leach solution.
68. The process of any one of embodiments 1-66, wherein the aqueous phase of step (c) is isolated from the mixture by gravity filtration.
69. The process of any one of embodiments 1-68, wherein the leach solution is substantially free of undissolved solids.
70. The process of any one of embodiments 1-40, further comprising:
   (d) contacting the leach solution of step (c) with a conjugate comprising a peptide and a polystyrene (PS) bead for a period of time under conditions that form a conjugate-metal ion complex, wherein the peptide is configured to selectively bind nickel and/or cobalt;
   (e) isolating the conjugate-metal ion complex of step (d);
   (f) eluting the metal ion(s) from the conjugate-metal ion complex by washing the complex with acid, thereby forming a solution;
   (g) transferring the solution of step (f) comprising the metal ions to an electrowinning circuit;
   (h) applying an electric current through the solution of step (f) comprising the metal ions;
   (i) collecting the resulting metal(s) on a surface of a cathode; and
   (j) recovering the metal(s) from the cathode.
71. The process of embodiment 70, wherein the peptide and PS bead are covalently attached.
72. The process of embodiment 70 or 71, wherein the peptide comprises a histidine residue.
73. The process of any one of embodiments 70-72, where the peptide comprises from 3 to 10 amino acid residues.
74. The process of any one of embodiments 70-73, wherein the peptide comprises:

—NH-X_{AA1}-X_{AA2}-His-,

wherein X_{AA1} and X_{AA2} are each independently an amino acid residue other than histidine.
75. The process of any one of embodiments 70-74, wherein the peptide comprises:

—NH-X_{AA1}-X_{AA2}-His-X_{AA3},

wherein X_{AA1}, X_{AA2}, and X_{AA2} are each independently an amino acid residue other than histidine.
76. The process of embodiment 74 or 75, wherein each X_{AA1} and X_{AA2} is independently a residue of glycine, alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or a-hydroxymethylserine.
77. The process of any one of embodiments 74-76, wherein —NH-X_{AA1}-X_{AA2}-His- is selected from the group consisting of: —NH-DAH, —NH-DTH, —NH-VIH, —NH-MDH, —NH-RFH, —NH-RTH, —NH-HSH, —NH-GGH, —NH-GKH, —NH-KGH, -NH-KKH, —NH-YYH, —NH-MNH, -NH-kGH, —NH-kGH, —NH-GkH, and —NH-kkH.
78. The process of any one of embodiments 75-77, wherein X_{A}M is a lysine residue.
79. The process of any one of embodiments 70-78, wherein the peptide has the structure:

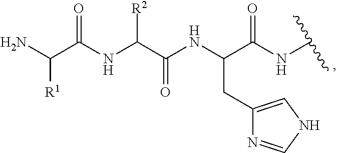

wherein R¹ and R² are each independently H or a side chain of an amino acid.
80. The process of embodiment 79, wherein the side chain is positively charged.
81. The process of embodiment 79 or embodiment 80, wherein the side chain comprises an amine or guanidine moiety.
82. The process of any one of embodiments 79-81, wherein R¹ and R² are each independently the side chain of a lysine, ornithine, arginine, or homoarginine residue.
83. The process of any one of embodiments 70-82, wherein the PS bead comprises a plurality of H₂N-PEG groups.
84. The process of embodiment 83, wherein the H₂N-PEG groups are on the surface of the PS bead.
85. The process of any one of embodiments 70-84, wherein the peptide is covalently linked to the PS bead.
86. The process of any one of embodiments 70-85, wherein one or more peptides are covalently linked to the PS bead
87. The process of embodiment 85 or 86, wherein the peptide is covalently linked to the PS bead by an amine of the PEG group.
88. The process of any one of embodiments 70-87, wherein the leach solution of step (d) comprises dissolved nickel, and optionally, cobalt.
89. The process of any one of embodiments 70-88, wherein the peptide is configured to selectively bind nickel.
90. The process of any one of embodiments 70-89, wherein the peptide has a selectivity for nickel over iron that is greater than 3:1.
91. The process of any one of embodiments 70-89, wherein the peptide has a selectivity for nickel over iron that is greater than 10:1.
92. The process of any one of embodiments 70-91, wherein the nickel is in the form of Ni^{II} ions.
93. The process of embodiment 92, wherein the peptide has a binding affinity for Ni^{II} of from about 1×10⁻¹⁶ to about 1×10⁻⁸ M⁻¹.
94. The process of any one of embodiments 70-93, wherein the leach solution of step (d) comprises a conjugate-Ni^{II} ion coordination complex.
95. The process of any one of embodiments 70-94, wherein greater than about 50% of the nickel in the leach solution is complexed to the conjugate after the period of time in step (d).
96. The process of any one of embodiments 70-95, wherein greater than about 80% of the nickel in the leach solution is complexed to the conjugate after the period of time in step (d).
97. The process of any one of embodiments 70-96, wherein the peptide is configured to selectively bind cobalt.
98. The process of any one of embodiments 70-97, wherein the peptide has a selectivity for cobalt over iron that is greater than 3:1.
99. The process of any one of embodiments 70-97, wherein the peptide has a selectivity for cobalt over iron that is greater than 10:1.
100. The process of any one of embodiments 70-99, wherein the cobalt is in the form of Co^{II} ions.
101. The process of embodiment 100, wherein the peptide has a binding affinity for Co^{II} of from about 1×10⁻¹⁶ to about 1×10⁻⁸ M⁻¹.
102. The process of any one of embodiments 70-101, wherein the leach solution of step (d) comprises a conjugate-Co^{II} ion coordination complex.
103. The process of any one of embodiments 70-102, wherein greater than about 50% of the cobalt in the leach solution is complexed to the conjugate after the period of time in step (d).
104. The process of any one of embodiments 70-103, wherein greater than about 80% of the cobalt in the leach solution is complexed to the conjugate after the period of time in step (d).
105. The process of any one of embodiments 70-104, wherein the contacting of step (d) comprises the application of heat and/or stirring.
106. The process of any one of embodiments 70-105, wherein the period of time in step (d) is from about 1 h to about 24 h.
107. The process of any one of embodiments 70-106, wherein step (e) isolating the conjugate-metal ion complex comprises filtering off the leach solution.
108. The process of any one of embodiments 70-107, wherein the acid has a pH ranging from about 2-4.
109. The process of any one of embodiments 70-108, wherein the solution of step (f) comprises greater than 60% of the nickel in the leach solution of step (a).
110. The process of any one of embodiments 70-109, wherein the solution of step (f) comprises greater than 80% of the nickel in the leach solution of step (a).
111. The process of any one of embodiments 70-110, wherein the nickel recovered from the cathode in step 0) represents greater than about 50% by weight of the nickel in the oxide ore.
112. The process of any one of embodiments 70-111, wherein the nickel recovered from the cathode in step 0) represents greater than about 75% by weight of the nickel in the oxide ore.
113. The process of any one of embodiments 70-112, wherein the cobalt recovered from the cathode in step 0) represents greater than about 50% by weight of the cobalt in the oxide ore.
114. The process of any one of embodiments 70-113, wherein the cobalt recovered from the cathode in step 0) represents greater than about 75% by weight of the cobalt in the oxide ore.
115. A conjugate comprising a peptide and a polystyrene (PS) bead.
116. The conjugate of embodiment 115, wherein the peptide and PS bead are covalently attached.
117. The conjugate of embodiment 115 or 116, wherein the peptide comprises a histidine residue.
118. The conjugate of any one of embodiments 115-117, wherein the peptide comprises from 3 to 10 amino acid residues.
119. The conjugate of any one of embodiments 115-118, wherein the peptide comprises the structure:

—NH-X_{AA1}-X_{AA2}-His-,

wherein X_{AA1} and X_{AA2} are each independently an amino acid residue other than histidine.
120. The conjugate of any one of embodiments 115-118, wherein the peptide comprises the structure:

—NH-X_{AA1}-X_{AA2}-His-X_{AA3},

wherein X_{AA1}, X_{AA2}, and X_{AA2} are each independently an amino acid residue other than histidine.
121. The conjugate of embodiment 119 or 120, wherein each X_{AA1} and X_{AA2} is independently a residue of glycine, alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or a-hydroxymethylserine.
122. The conjugate of any one of embodiments 119-121, wherein —NH-X_{AA1}-X_{AA2}-His- is selected from the group consisting of: -HN-DAH, -HN-DTH, -HN-VIH, -HN-MDH, -HN-RFH, -HN-RTH, -HN-HSH, -HN-GGH, -HN-GKH, -HN-KGH, -HN-KKH, -HN-YYH, -HN-MNH, -HN-kGH, -HN-kGH, -HN-GkH, and -HN-kkH.
123. The conjugate of any one of embodiments 120-122, wherein X_{AA3} is a lysine residue.
124. The conjugate of any one of embodiments 115-123, having the structure:

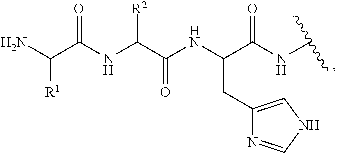

wherein R¹ and R² are each independently H or a side chain of an amino acid.
125. The conjugate of embodiment 10, wherein the side chain is positively charged.
126. The conjugate of embodiment 124 or embodiment 125, wherein the side chain comprises an amine or guanidine moiety.
127. The conjugate of any one of embodiments 124-126, wherein R¹ and R² are each independently the side chain of a lysine, ornithine, arginine, or homoarginine residue.
128. The conjugate of any one of embodiments 115-127, wherein the peptide is configured to selectively bind nickel.
129. The conjugate of any one of embodiments 115-128, wherein the peptide has a selectivity for nickel over iron that is greater than 3:1.
130. The conjugate of any one of embodiments 115-128, wherein the peptide has a selectivity for nickel over iron that is greater than 10:1.
131. The conjugate of any one of embodiments 115-130, wherein the nickel is in the form of Ni^{II} ions.
132. The conjugate of any one of embodiments 115-131, wherein the peptide has a binding affinity for Ni^{II} of from about 1×10⁻¹⁶ to about 1×10⁻⁸ M⁻¹.
133. The conjugate of any one of embodiments 115-132, wherein the peptide is configured to selectively bind cobalt.
134. The conjugate of any one of embodiments 115-133, wherein the peptide has a selectivity for cobalt over iron that is greater than 3:1.
135. The conjugate of any one of embodiments 115-133, wherein the peptide has a selectivity for cobalt over iron that is greater than 10:1.
136. The conjugate of any one of embodiments 115-135, wherein the cobalt is in the form of Co^{II} ions.
137. The conjugate of any one of embodiments 115-136, wherein the peptide has binding affinity for Co^{II} is from about 1×10⁻¹⁶ to about 1×10⁻⁸ M⁻¹.
138. The conjugate of any one of embodiments 115-137, wherein the PS bead comprises a plurality of H₂N-PEG groups.
139. The conjugate of embodiment 138, wherein the H₂N-PEG groups are on the surface of the PS bead.
140. The conjugate of any one of embodiments 115-139, wherein the peptide is covalently linked to the PS bead.
141. The conjugate of any one of embodiments 115-139, wherein one or more peptides are covalently linked to the PS bead
142. The conjugate of embodiment 140 or 141, wherein the peptide is covalently linked to the PS bead by an amine of the PEG group.

### EXAMPLES

Leaching experiments are described herein and include closed-system batch incubations and open-system flow-through incubations. The open-system incubations will be used to overcome limitations related to the accumulation of reaction products.

### Example 1. Closed-System Batch Incubations

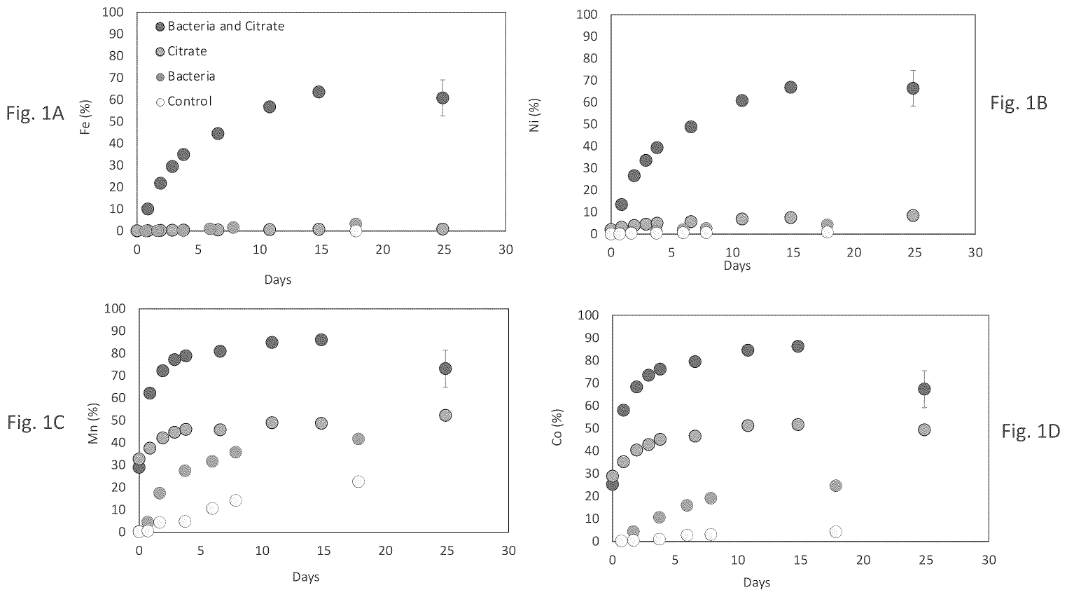

Closed-system, batch incubation experiments were conducted similar to that described in Crowe, S. A., et al. “Alteration of iron-rich lacustrine sediments by dissimilatory iron-reducing bacteria.” Geobiology 5.1 (2007): 63-73, which is incorporated by reference in its entirety herein, with additional steps and modifications. Experiments herein were conducted in sealed vessels (e.g., mason jars or serum bottles) from which air has been purged with N₂ gas and which comprise four components: 1) synthetic or natural, nickel and cobalt containing, Fe oxyhydroxide minerals including laterite soils; 2) an aqueous medium containing salts and nutrients (tabulated below); 3) a substrate (electron donor); and 4) an organism or assemblages of organisms. Incubation experiments are conducted over the course of multiple days to several months. The aqueous phase is sampled and separated from the oxyhydroxides by filtration or centrifugation. Solution chemistry, including Fe, Ni, and Co concentrations is monitored (sampled and preserved in 2% HNO₃ for downstream analysis by ICP-OES or ICP-MS) as a function of time to determine the extent of Fe mineral dissolution and associated release of Ni and Co to the aqueous phase (as shown in FIGS. 1A-1D).

Benchmark aqueous medium composition (varied to attain a minimal salt concentration)
NH₄Cl: 28 mM
KCl: 1.39 mM
CaCl₂): 0.68 mM
NaClO₄: 50 mM

### Example 2. Batch Bioleaching in Laterites with S. Putrefaciens

Lactate as the electron donor was supplied to S. putrefaciens as shown in FIGS. 1A-ID, microbial iron reduction is effective at reducing Fe minerals from laterites. Over the course of 17 days, S. putrefaciens reductively dissolved 3% and 42% of the total Fe and Mn in the laterite material, respectively. In response, approximately 4% and 25% of the Ni and Co were released to solution, respectively. This corresponds to Ni and Co concentrations of 27 μM and 6 μM, respectively. Such concentrations, in a simple circumneutral pH solution, are amenable to downstream ion exchange capture and recovery.

### Example 3. Batch Bioleaching in Laterites with S. Putrefaciens and 50 mM Citrate

Lactate as the electron donor was supplied to S. putrefaciens. As shown in FIGS. 1A-1D, microbial iron reduction is effective at reducing Fe minerals from laterites. Over the course of 25 days, S. putrefaciens reductively dissolved about 65% and 85% of the total Fe and Mn in the laterite material, respectively. In response, approximately 65% and 85% of the Ni and Co were released to solution, respectively. This corresponds to Ni and Co concentrations of 430 μM and 22 μM, respectively. Such concentrations, in a simple circumneutral pH solution, are amenable to downstream ion exchange capture and recovery.

### Example 4. Batch Bioleaching in Laterites with Natural Community and 50 mM Citrate

Lactate as the electron donor was supplied to S. putrefaciens. As shown in FIGS. 1A-ID, microbial iron reduction is effective at reducing Fe minerals from laterites. Over the course of 25 days, S. putrefaciens reductively dissolved about 1% and 25% of the total Fe and Mn in the laterite material, respectively. In response, approximately 8% and 52% of the Ni and Co were released to solution, respectively. This corresponds to Ni and Co concentrations of 54 μM and 13 μM, respectively. Such concentrations, in a simple circumneutral pH solution, are amenable to downstream ion exchange capture and recovery.

### Example 5. Open System Flow-through Incubations

As shown in FIG. 2, Open-system, flow-through incubations were conducted similar to those described in Roden, Eric E., Matilde M. Urrutia, and Carroll J. Mann. “Bacterial reductive dissolution of crystalline Fe (III) oxide in continuous-flow column reactors.” Applied and Environmental Microbiology 66.3 (2000): 1062-1065, which is incorporated by reference herein in its entirely, with additional components and steps as described for the closed-system incubations above in Example 1. Synthetic and/or natural Fe oxyhydroxide minerals including laterite soils were packed into column reactors through which the aqueous medium, electron donor, and organisms were pumped. Solution chemistry, including Fe, Ni, Co and Mn concentrations were monitored in the column effluent as a function of time (sampled and preserved in 2% HNO₃ for downstream analysis by ICP-OES or ICP-MS) to determine the extent of Fe mineral dissolution and associated dissolution of Ni and Co.

### Example 6. Flow-Through Bioleaching in Laterites with S. putrefaciens and 50 mM Citrate

Lactate as the electron donor was supplied to S. putrefaciens. As shown in FIG. 2, microbial iron reduction is effective at reducing Fe minerals from laterites. Over the course of 90 days, S. putrefaciens reductively dissolved about 100% of the total Fe and Mn in the laterite material, respectively. In response, approximately 100% of the Ni and Co were released to solution, respectively. This corresponds to Ni and Co concentrations up to 112 μM and 56 μM, respectively. Such concentrations, in a simple circumneutral pH solution, are amenable to downstream ion exchange capture and recovery.

### Example 7. Flow-Through Bioleaching in Laterites with S. putrefaciens

Lactate as the electron donor is supplied to S. putrefaciens. Microbial iron reduction is effective at reducing Fe minerals from laterites. Over the course of 300 days, S. putrefaciens reductively dissolves about 100% and 100% of the total Fe and Mn in the laterite material, respectively. In response, approximately 100% and 100% of the Ni and Co is released to solution, respectively. This corresponds to Ni and Co concentrations up to 112 μM and 56 μM, respectively. Such concentrations, in a simple circumneutral pH solution, are amenable to downstream ion exchange capture and recovery.

### Example 8. Bioleaching in Laterites with Geobacter Metallireducens

Acetate as the electron donor is supplied to Geobacter metallireducens. Microbial iron reduction is effective at reducing Fe minerals from laterites. Over the course of 1-20 days, Geobacter metallireducens reductively dissolves 5-50% and 40-100% of the total Fe and Mn in the laterite material, respectively. In response, approximately 4-40% and 25-100% of the Ni and Co is released to solution, respectively. This corresponds to Ni and Co concentrations of 25-250 μM and 6-24 μM, respectively. Such concentrations, in a simple circumneutral pH solution, are amenable to downstream ion exchange capture and recovery.

### Example 9. Bioleaching in Laterites with Clostridium celerecrescens

Glucose as the electron donor is supplied to Clostridium celerecrescens. Over the course of two months, Clostridium celerecrescens, reduces 10-100% of the total Fe in the laterite material with glucose. This yields a corresponding dissolution of 10-100% of the laterite Ni and Co, produces leachates with Ni and Co concentrations of about 64-640 μM, and 3-25 μM, respectively. Such concentrations, in a simple circumneutral pH solution, are then recovered using ion exchange chromatography.

### Example 10. Bioleaching in Laterites with a Mixed Consortium of S. putrefaciens, Clostridium Celerecrescens, and Acidobacterium Capsulatum

Molasses is supplied to the consortium. Over the course of one week, the consortium reduces 20% of the total Fe in the laterite material with lactate. Assuming Ni and Co dissolution congruent with Fe reduction, this yields a corresponding dissolution of 20% of the laterite Ni and Co, which at molar Fe:Ni and Fe:Co ratios of 0.026 and 0.0009 in these materials produces leachates with Ni and Co concentrations of 600 μM, and 16 μM, respectively. Such concentrations, in a simple circumneutral pH solution, are amenable to downstream ion exchange capture and recovery.

### Example 11. Bioleaching in Laterites with S. putrefaciens Mutant

Lactate as the electron donor is supplied to S. putrefaciens mutant. Microbial iron reduction is effective at reducing Fe minerals from laterites. Over the course of 1-20 days, S. putrefaciens mutant reductively dissolves 5-50% and 40-100% of the total Fe and Mn in the laterite material, respectively. In response, approximately 4-40% and 25-100% of the Ni and Co is released to solution, respectively. This corresponds to Ni and Co concentrations of 25-250 μM and 6-24 μM, respectively. Such concentrations, in a simple circumneutral pH solution, are amenable to downstream ion exchange capture and recovery.

### Example 12: Metal Recovery

A pregnant leach solution containing Fe²⁺ and Ni²⁺ is first passed through an ion-exchange fixed-bed column to scavenge other metals, if present. The resulting solution is then passed through the disclosed ion exchange (IX) conjugate comprising a peptide and PS beads in which columns charged with the conjugate are fixed on a carousel. An acidified electrolyte solution, manufactured to correspond to a nickel electrowinning solution is used as a stripping solution. Impurities are rejected into the raffinate, with nickel recovered in the eluate. The eluate is transferred to an electrowinning circuit and current is applied. Nickel is collected on the surface of a cathode and recovered.

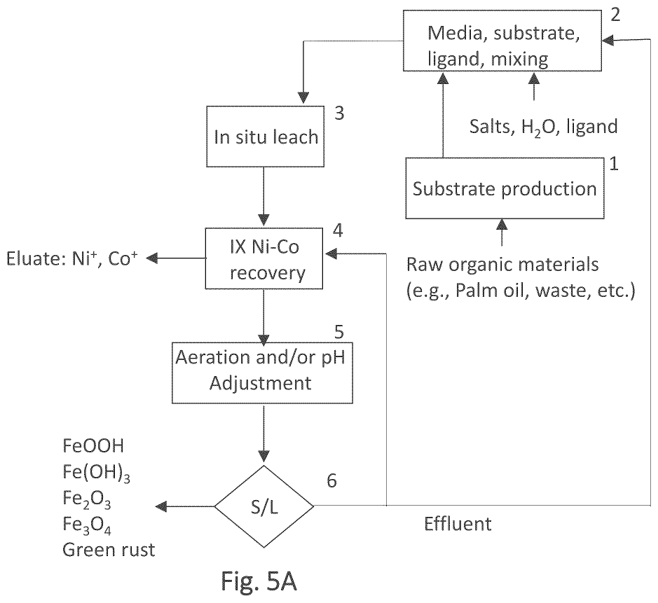

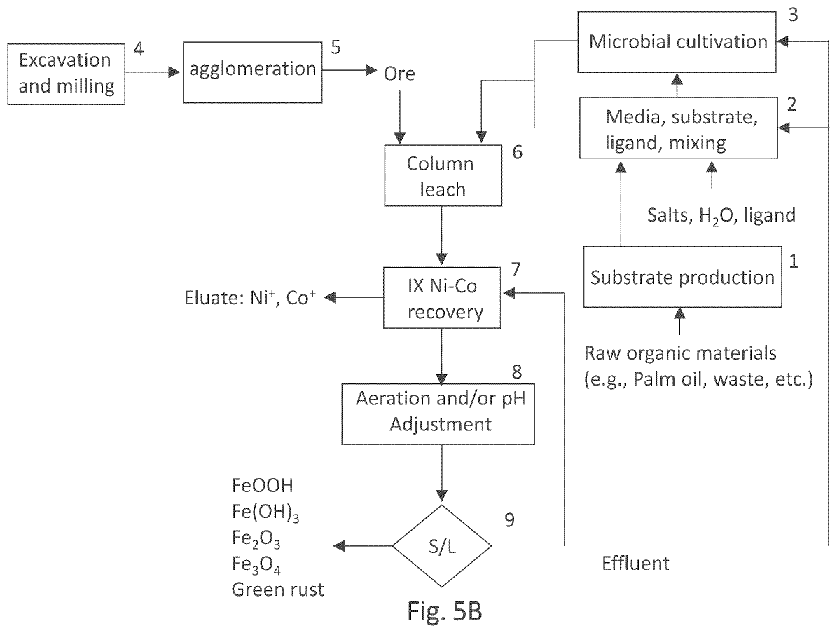

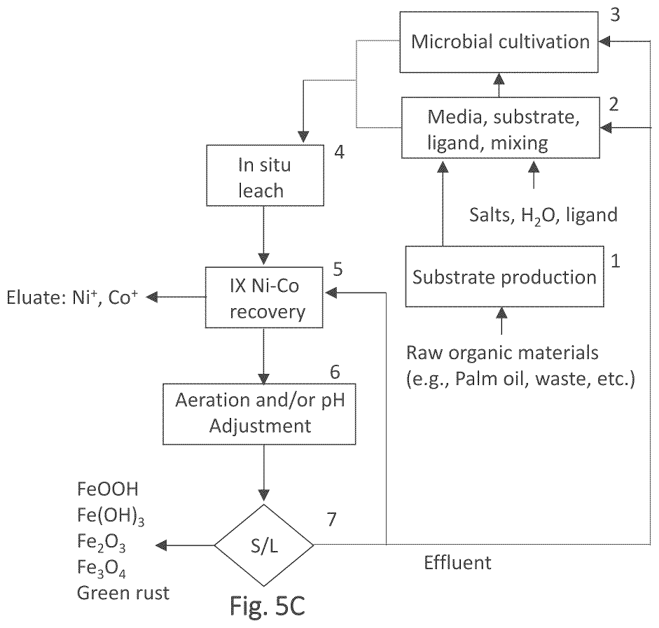

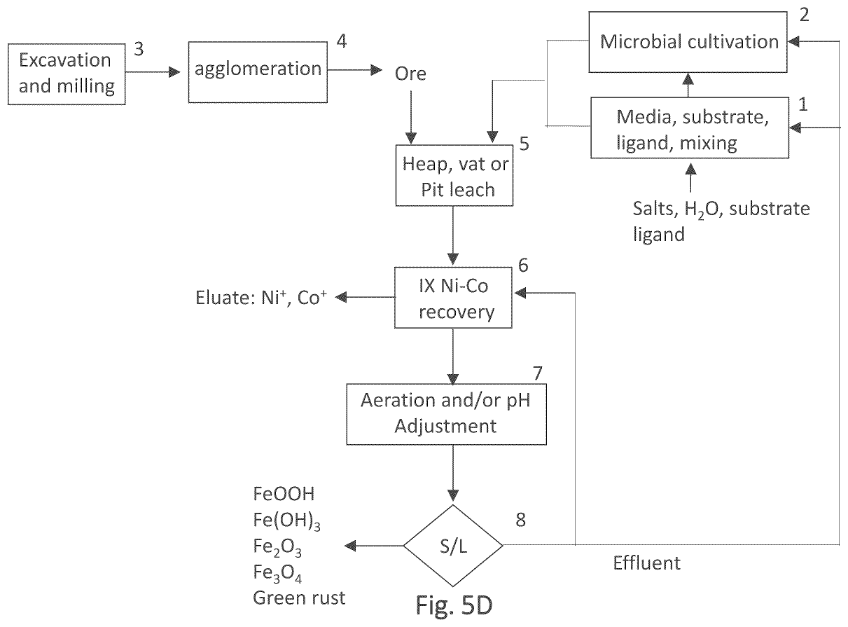

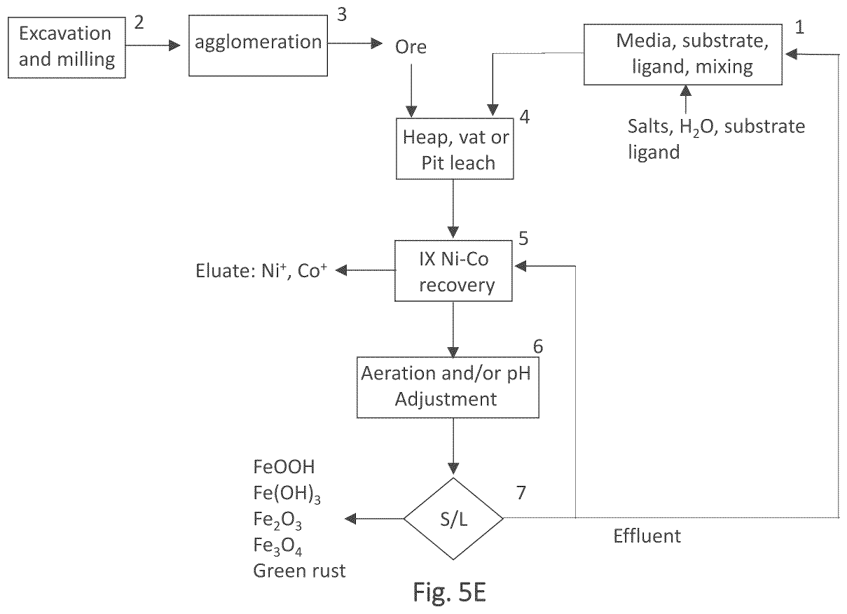

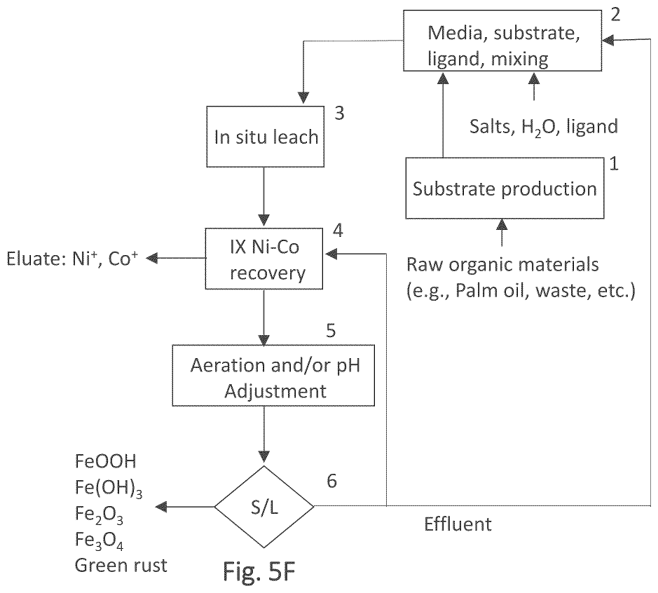

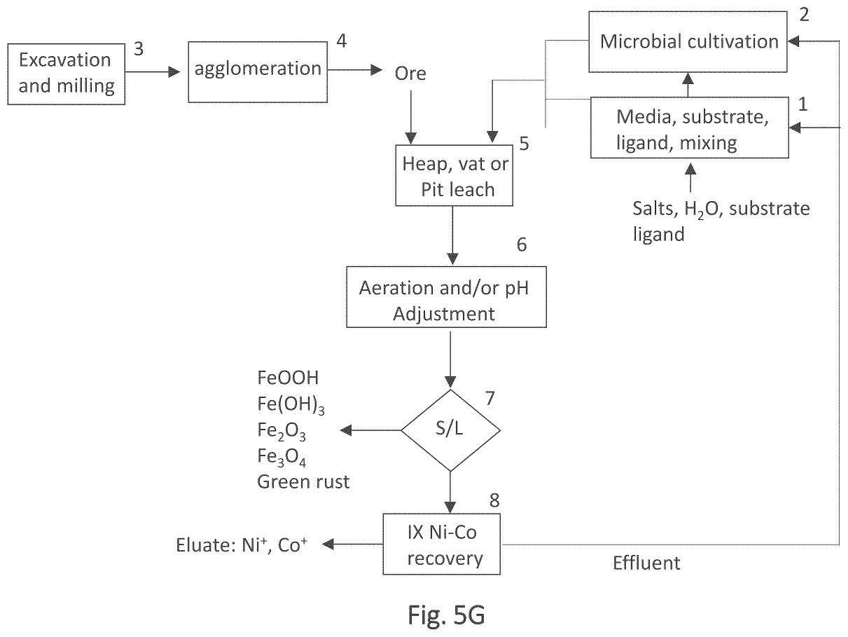

### Example 13: Leaching and Metal Recovery Via Circuit Configuration 1 (FIG. 5A

1) Substrates (e.g. volatile fatty acids) are produced through the fermentation of complex organic materials like palm oil or municipal sewage; 2) substrates from 1 are mixed with salts, water and ligands; 3) substrate containing mixture is irrigated over laterite soils without excavation and reductive leaching takes place directly within the soil pore-space; 4) leachate is collected from groundwater and passed through ion exchange circuit to recover nickel, cobalt, and other metals; 5) leachate is aerated to oxidize iron and/or pH adjusted to induce precipitation; 6) iron precipitates are separated from leachate and disposed of so that the effluent can be recycled.

### Example 14: Leaching and Metal Recovery Via Circuit Configuration 2 (FIG. 5B

1) Substrates (e.g. volatile fatty acids) are produced through the fermentation of complex organic materials like palm oil or municipal sewage; 2) substrates from 1 are mixed with salts, water and ligands; 3) Specific microorganisms are cultivated through industrial fermentation and then inoculated into the substrate containing mixture, 4) laterite soils are excavated, and milled and ground if needed; 5) the soils are agglomerated, if need be, and then transported to the leaching columns; 6) the ore is loaded into leaching columns and the inoculated substrate containing mixture is gravity fed or pumped through the columns; 7) leachate is collected from column outflow and passed through ion exchange circuit to recover nickel, cobalt, and other metals; 8) leachate is aerated to oxidize iron and/or pH adjusted to induce precipitation; 9) iron precipitates are separated from leachate and disposed of so that the effluent can be recycled.

### Example 15: Leaching and Metal Recovery Via Circuit Configuration 3 (FIG. 5C

1) Substrates (e.g. volatile fatty acids) are produced through the fermentation of complex organic materials like palm oil or municipal sewage; 2) substrates from 1 are mixed with salts, water and ligands; 3) Specific microorganisms are cultivated through industrial fermentation and then inoculated into the substrate containing mixture; 3) the inoculated substrate containing mixture is irrigated over laterite soils without excavation and reductive leaching takes place directly within the soil pore-space; 4) leachate is collected from groundwater and passed through ion exchange circuit to recover nickel, cobalt, and other metals; 6) leachate is aerated to oxidize iron and/or pH adjusted to induce precipitation; 7) iron precipitates are separated from leachate and disposed of so that the effluent can be recycled.

### Example 16: Leaching and Metal Recovery Via Circuit Configuration 4 (FIG. 5D

1) substrates are mixed with salts, water and ligands; 2) specific microorganisms are cultivated through industrial fermentation and then inoculated into the substrate containing mixture, 3) laterite soils are excavated, and milled and ground if needed; 4) the soils are agglomerated, if need be, and then transported to the leaching pad, pit or vat; 5) the ore is piled into a heap, pit, or vat and then the inoculated substrate containing mixture is irrigated onto the heap; 6) leachate is collected from the heap base and passed through an ion exchange circuit to recover nickel, cobalt, and other metals; 7) leachate is aerated to oxidize iron and/or pH adjusted to induce precipitation; 8) iron precipitates are separated from leachate and disposed of so that the effluent can be recycled.

### Example 17: Leaching and Metal Recovery Via Circuit Configuration 5 (FIG. 4E

1) substrates are mixed with salts, water and ligands; 2) laterite soils are excavated, and milled and ground if needed; 3) the soils are agglomerated, if need be, and then transported to the leaching pad, pit or vat; 4) the ore is piled into a heap, pit or vat and then the substrate containing mixture is irrigated onto the heap; 5) leachate is collected from the heap base and passed through an ion exchange circuit to recover nickel, cobalt, and other metals; 6) leachate is aerated to oxidize iron and/or pH adjusted to induce precipitation; 7) iron precipitates are separated from leachate and disposed of so that the effluent can be recycled.

### Example 18: Leaching and Metal Recovery Via Circuit Configuration 6 (FIG. 4F

1) Substrates (e.g. volatile fatty acids) are produced through the fermentation of complex organic materials like palm oil or municipal sewage; 2) substrates are mixed with salts, water and ligands; 3) laterite soils are excavated, and milled and ground if needed, then transported to the leaching pad; 4) the ore is piled into a heap and then the substrate containing mixture is irrigated onto the heap; 5) leachate is collected from the heap base and passed through an ion exchange circuit to recover nickel, cobalt, and other metals; 6) leachate is aerated to oxidize iron and/or pH adjusted to induce precipitation; 7) iron precipitates are separated from leachate and disposed of so that the effluent can be recycled.

### Example 19: Leaching and Metal Recovery Via Circuit Configuration 6 (FIG. 4G

1) substrates are mixed with salts, water and ligands; 2) specific microorganisms are cultivated through industrial fermentation and then inoculated into the substrate containing mixture, 3) laterite soils are excavated, and milled and ground if needed; 4) the soils are agglomerated, if need be, and then transported to the leaching pad, pit or vat; 5) the ore is piled into a heap, pit, or vat and then the inoculated substrate containing mixture is irrigated onto the heap; 6) leachate is aerated to oxidize iron and/or pH adjusted to induce precipitation; 7) iron precipitates are separated from leachate and disposed of so that the effluent can be recycled. 8) leachate is collected from the heap base and passed through an ion exchange circuit to recover nickel, cobalt, and other metals.

### Example 20: Iron Removal Through Aeration and pH Adjustment

1) leach solution is aerated to oxidize some or all ferrous iron to ferric iron; 2) pH is raised through addition of based to a pH of greater than 10 to induce precipitation; 3) resulting precipitate is separated from leach through gravity settling, filtrations, and/or any other physical means. Laboratory tests show removal of greater than 90% of the Fe from a typical leach solution through this process.

The patents and publications listed herein describe the general skill in the art and are hereby incorporated by reference in their entireties for all purposes and to the same extent as if each was specifically and individually indicated to be incorporated by reference. Nothing herein is to be construed as an admission that the present invention is not entitled to antedate such publication by virtue of prior invention. In describing embodiments of the present application, specific terminology is employed for the sake of clarity. However, the invention is not intended to be limited to the specific terminology so selected. Nothing in this specification should be considered as limiting the scope of the present invention. Many modifications and other embodiments of the inventions set forth herein will come to mind to one skilled in the art to which these inventions pertain having the benefit of the teachings presented in the foregoing descriptions and the associated drawings. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

1-142. (canceled)

143. A leaching process for recovering nickel, manganese, and/or cobalt from an oxide ore, the process comprising:
(a) combining (i) one or more microorganisms, (ii) a substrate mixture, (iii) the oxide ore, (iv) a ligand, (v) an optional electron shuttle, and (vi) an aqueous phase to form a mixture;
(b) maintaining the mixture of step a) for a pre-determined period of time under conditions sufficient to at least partially reduce an amount of ferric iron in the ore to ferrous iron, wherein the ferrous iron, nickel, cobalt, and manganese are solubilized in the aqueous phase; and
(c) isolating the aqueous phase as a leach solution comprising the ferrous iron and an amount of dissolved nickel, manganese, and/or cobalt, from the mixture.

144. The process of claim 143, wherein the one or more microorganisms of step (a) are added to a slurry comprising substrate mixture, the ore, the ligand, and the aqueous phase.

145. The process of claim 143, wherein the one or more microorganisms of step (a) are derived from the ore.

146. The process of claim 143, wherein the one or more microorganisms produce an enzyme or enzyme system selected from the group consisting of CymA, DFE, DmkA/DmkB, EetA/EetB, FmnA/FmnB, GACE, MtrA/MtrA/MtrC, Ndh2, OmcF/OmcS/OmcZ, PplA, and T4ap, or combinations thereof.

147. The process claim 143, wherein the one or more microorganisms are selected from the group consisting of Thermincola potens JR (NC_014152.1), Listeria monocytogenes (NC_003210.1), Clostridium celerecrescens (NZ_PGET01000001.1), Bacillus infernus, Bacillus subterraneus, Bacillus pseudormus MC02, Bacillus subterraneus, Thermoanaerobacter siderophilus (NZ_CM001486.1), Carboxydothermus ferrireducens (NZ_A TYG01000001.1), Carboxydothermus siderophilus, Carboxydothermus pertinax (NZ_BDJK01000055.1), Carboxydocella thermautotrophica (NZ_CP028491.1), Moorella humiferrea (NZ_PVXM01000006.1), Geosporobacter ferrireducens IRF9, Thermotalea metallivorans B2-1(7), Pelosinus fermentans, Thermotoga maritima (NC_023151.1), Sinirhodobacter ferrireducens (NZ_SAVB01000001.1), Acidiphilium SJH, Acidiphilium PK40, Acidiphilium PK46, Acidiphilium KPW14, Acidiphilium cryptum JF-5, Acidocella facilis, Acidocella M21, Acidocella PFBC, Acidocella PWB4, Shewanella putrefaciens (NZ_CP066370.1), Shewanella oneidensis MR-1 (NC_004347.2), Shewanella alga strain BrY (NZ_CP046378.1), Shewanella amazonensis sp. nov., Shewanella putrefaciens IR-1, Shewanella sp. HN-41, Shewanella putrefaciens CN-32, Shewanella putrefaciens 200R, Shewanella oneidensis MR-1, Shewanella baltica W3-6-1, Shewanella sp.PV-4, Shewanella peizotolerans WP3, Shewanella decolorationis, Shewanella frigidimarina, Shewanella gelidimarina, Shewanella loihica, Shewanella pealeana, Serratia plymuthica (NC_015567.1), Serratia fonticola (NZ_CP011254.1), Aeromonas hydrophila (NZ_CP050851.1), Klebsiella oxytoca (NZ_CP033844.1), Ferrimonas balearica (NC_014541.1), Frateuria-like isolate WJ2, Desulfovibrio ferrophilus (NZ_AP017378.1), Desulfuromusa ferrireducens, Desulfuromonas svalbardensis, Desulfuromonas acetoxidans (JABWTG01), Geobacter sulfurreducens (NC_002939.5), Geobacter bemidjiensis(NC_011146.1), Geobacter metallireducens (NC_007517.1), Geobacter pelophilus strain Dfr2 (NZ_JAHCVJ010000001.1), Geobacter daltonii (NC_011979.1), Geobacter chapellei (NZ_JAHDYS010000001.1), Geobacter psychrophilus sp. nov, Geobacter bremensis sp. nov., Geobacter lovleyi sp. nov. strain SZ, Geobacter luticola, Geobacter pickeringii, Geobacter argillaceus, Geobacter uraniireducens, Desulfosediminicola ganghwensis (NZ_CP050699.1), Desulfosediminicola flagellatus (NZ_CP050698.1), Anaeromyxobacter dehalogenans (NC_011891.1), Anaeromyxobacter Strain FAc12, Geoalkalibacter subterraneus (NZ_CP010311.1), Geoalkalibacter ferrihydriticus (NZ_FNGU01000001.1), Rhodoferax ferrireducens (NC_007908.1), Ferribacterium limneticum (NZ_CP075189.1), Geothrix fermentans (NZ_KE386810.1), Geovibrio ferrireducens, Deferribacter thermophilus, Pyrobaculum islandicum (NC_008701.1), Pyrodictium abyssi, Methanopyrus kandleri (NC_003551.1), Archaeoglobus fulgidus (NZ_CP006577.1), Pyrococcus furiosus (NZ_CP023154.1), Methanococcus thermolithotrophicus (NZ_AQXV01000055.1), Ferroglobus placidus (NC_013849.1), Geoglobus acetivorans sp. nov., Acidobacterium capsulatum, Acidobacterium PK35, Acidobacterium RIT23, Acidobacterium WJ7, Telmatospirillum (Alphaproteobacterial Genus) (PRJNA561022), Sideroxydans lithotrophicus ES-1, Magnetospirillum magneticum AMB-1, Candidatus Tectomicrobia, Candidatus Zixibacteria, Candidatus Tectomicrobia, Candidatus Dadabacteria, Candidatus Handelsmanbacteria, Carboxydothermus hydrogenoformans, Carboxydothermus islandicus, Telmatospirillum siberiense, Nitrosococcus halophilus, Skermanella stibiiresistens, Insolitispirillum peregrinum, Magnetospirillum magneticum, and mixtures and/or genetic mutants thereof.

148. The process of claim 143, wherein step a) comprises combining (i) one or more microorganisms, (ii) a substrate mixture, (iii) the oxide ore, (iv) the ligand, (v) an electron shuttle, and (vi) an aqueous phase to form a mixture, and the electron shuttle comprises one or more flavins, quinones, humic acids, fulvic acids, biochar, or a combination thereof.

149. The process of claim 143, wherein the one or more microorganisms is a Shewanella spp.

150. The process of claim 143, wherein the microorganism possesses one or more mutations, and at least one of the mutations enhances the microorganism's ability to reduce iron.

151. The process of claim 143, wherein the substrate mixture comprises a reducing agent and/or carbonaceous waste products, elemental sulfur, substances containing partially reduced sulfur forms, molasses, sugar, beet sugar, cane sugar, glucose derived from palm oil, dextrose hydrolyzed from corn, dextrose hydrolyzed from cornstarch, sucrose-containing materials, fermentation products thereof, acetate lactate, or mixtures thereof.

152. The process of claim 143, wherein the oxide ore is a laterite ore.

153. The process of claim 143, wherein the oxide ore comprises:
(a) from about 1% to about 90% by weight of one or more ferric iron minerals; and/or
(b) from about 5% to about 20% by weight iron.

154. The process claim 143, wherein the oxide ore comprises about 0.1% to about 15% by weight nickel, about 0.1% to about 5% by weight nickel, or about 0.1% to about 2.5% by weight nickel.

155. The process of claim 143, wherein the nickel in the oxide ore is associated with (a) one or more ferric iron minerals, quartz, manganese, and/or magnesium silicates or (b) ferric iron (oxy)hydroxides or α-FeO·OH (goethite).

156. The process of claim 143, wherein the oxide ore comprises about 0.01% to about 2.0% by weight cobalt or about 0.01% to about 0.5% by weight cobalt.

157. The process of claim 143, wherein the ligand comprises an organic acid including but not restricted to the group consisting of malic acid, lactic acid, gluconic acid, pyruvic acid, succinic acid, ketoglutaric acid, oxalic acid, fumaric acid, citric acid, acetic acid, malonic acid, salicylic acid, acetohydroxamic acid, mugineic acid, benzoic acid, hydroxamic acid, and a combination thereof.

158. The process of claim 143, wherein the conditions of step (b) comprise one or more of (a) maintaining the mixture at a temperature from about 5° C. to about 75° C., (b) maintaining the mixture at an absolute pressure of about one atmosphere, (c) maintaining the aqueous phase at a pH of from about 5 to 9, and (d) agitating the mixture.

159. The process of claim 143, wherein the reduction of ferric iron to ferrous iron is carried out under anoxic or substantially anoxic conditions.

160. The process of claim 143, wherein (i) about 5% to about 100% by weight of the nickel in the ore is solubilized in the aqueous phase after step (b) and/or (ii) about 5% to about 100% by weight of the cobalt in the ore is solubilized in the aqueous phase after step (b).

161. The process of claim 160, wherein the aqueous phase of step (c) is isolated from the mixture by sedimentation of the undissolved solids and decantation of the leach solution or by gravity filtration.

162. The process of claim 143, further comprising:
(d) contacting the leach solution of step (c) with a conjugate comprising a peptide and a polystyrene (PS) bead for a period of time under conditions that form a conjugate-metal ion complex, wherein the peptide is configured to selectively bind nickel, manganese, and/or cobalt;
(e) isolating the conjugate-metal ion complex of step (d);
(f) eluting the metal ion(s) from the conjugate-metal ion complex by washing the complex with acid, thereby forming a solution;
(g) transferring the solution of step (f) comprising the metal ions to an electrowinning circuit;
(h) applying an electric current through the solution of step (f) comprising the metal ions;
(i) collecting the resulting metal(s) on a surface of a cathode; and
(j) recovering the metal(s) from the cathode.

163. The process of claim 162, where the peptide comprises from 3 to 30 amino acid residues.

164. The process of claim 162, wherein the peptide comprises:
—NH-X_{AA1}-X_{AA2}-His-,
wherein X_{AA1} and X_{AA2} are each independently an amino acid residue other than histidine.

165. The process of claim 162, wherein the peptide comprises:
—NH-X_{AA1}-X_{AA2}-His-X_{AA3},
wherein X_{AA1}, X_{AA2}, and X_{AA3} are each independently an amino acid residue other than histidine.

166. The process of claim 164, wherein each X_{AA1} and X_{AA2} is independently a residue of glycine, alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or a-hydroxymethylserine.

167. The process of claim 164, wherein —NH-X_{AA1}-X_{AA2}-His- is selected from the group consisting of: —NH-DAH, —NH-DTH, —NH-VIH, —NH-MDH, —NH-RFH, —NH-RTH, —NH-HSH, -NH-GGH, —NH-GKH, —NH-KGH, -NH-KKH, —NH-YYH, —NH-MNH, —NH-kGH, —NH-kGH, -NH-GkH, and —NH-kkH.

168. The process of claim 165, wherein X_{AA3} is at least one lysine residue.

169. The process of claim 162, wherein the peptide has the structure: 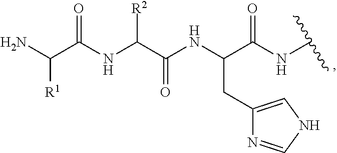
wherein R¹ and R² are each independently H or a side chain of an amino acid.

170. The process of claim 169, wherein R¹ and R² are each independently the side chain of a lysine, ornithine, arginine, or homoarginine residue.

171. The process of claim 162, wherein the polystyrene bead comprises a plurality of H₂N-PEG groups.


SEQUENCE LISTING (5 sequences)

SEQ ID NO: 1 - protein, 82 aa, Streptomyces coelicolor
MAHFMDVHRGMHGITSDQLHQAHQADLAVEKDENVHFEQAWADPASGTIYCLSEGPSAEAVQRVHERAGHKADEIHEVPLSA

SEQ ID NO: 2 - protein, 6 aa, synthetic construct
HHHHHH

SEQ ID NO: 3 - protein, 41 aa, synthetic construct
ECECECECECECECECECECECECECECECECECECECECG

SEQ ID NO: 4 - protein, 36 aa, Escherichia coli
  SITE 1: note = N-terminus is acetylated
  SITE 36: note = C-terminus is amidated
KGCHLDAQMIADAAPRLPLDDNGILFIENVGNLVCP

SEQ ID NO: 5 - protein, 10 aa, Escherichia coli
  SITE 1: note = N-terminus is amidated
  SITE 10: note = C-terminus is amidated
MCTTCGCGEG